SEQUENCE LISTING (20 sequences)

SEQ ID NO: 1 - protein, 16 aa, Artificial Sequence
  linker amino acid sequence
GGGSGGGSGGGSGGGS

SEQ ID NO: 2 - protein, 103 aa, Artificial Sequence
  Murine IgG1, CAmino acid sequence for CH1 domain
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCG

SEQ ID NO: 3 - protein, 221 aa, Artificial Sequence
  Murine IgG1,  Amino acid sequence for <hinge>-CH2-CH3 portion (FcH)
CKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 4 - protein, 108 aa, Artificial Sequence
  Murine IgG2, amino acid sequence for CH1 domain
AKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCP

SEQ ID NO: 5 - protein, 222 aa, Artificial Sequence
  Murine IgG2, Amino acid sequence for <hinge>-CH2-CH3 portion (FcH)
PCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 6 - protein, 96 aa, Artificial Sequence
  Murine IgG3, Amino acid sequence for CH1 domain
TTTAPSVYPLVPGCSDTSGSSVTLGCLVKGYFPEPVTVKWNYGALSSGVRTVSSVLQSGFYSLSSLVTVPSSTWPSQTVICNVAHPASKTELIKRI

SEQ ID NO: 7 - protein, 233 aa, Artificial Sequence
  Murine IgG3, Amino acid sequence for <hinge>-CH2-CH3 portion (FcH)
EPRIPKPSTPPGSSCPAGNILGGPSVFIFPPKPKDALMISLTPKVTCVVVDVSEDDPDVHVSWFVDNKEVHTAWTQPREAQYNSTFRVVSALPIQHQDWMRGKEFKCKVNNKALPAPIERTISKPKGRAQTPQVYTIPPPREQMSKKKVSLTCLVTNFFSEAISVEWERNGELEQDYKNTPPILDSDGTYFLYSKLTVDTDSWLQGEIFTCSVVHEALHNHHTQKNLSRSPGK

SEQ ID NO: 8 - protein, 104 aa, Artificial Sequence
  Human IgG1, Amino acid sequence for CH1 domain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCD

SEQ ID NO: 9 - protein, 226 aa, Artificial Sequence [sequence omitted]

SEQ ID NO: 10 - protein, 102 aa, Artificial Sequence
  Human IgG2, Amino acid sequence for CH1 domain
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKC

SEQ ID NO: 11 - protein, 224 aa, Artificial Sequence
  Human IgG2, Amino acid sequence for <hinge>-CH2-CH3 portion (FcH)
CVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 12 - protein, 112 aa, Artificial Sequence
  Human IgG3, Amino acid sequence for CH1 domain
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCP

SEQ ID NO: 13 - protein, 265 aa, Artificial Sequence
  Human IgG3, Amino acid sequence for <hinge>-CH2-CH3 portion (FcH)
RCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 14 - protein, 103 aa, Artificial Sequence
  Human IgG4, Amino acid sequence for CH1 domain
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVSPNMV

SEQ ID NO: 15 - protein, 224 aa, Artificial Sequence
  Human IgG4, Amino acid sequence for <hinge>-CH2-CH3 portion (FcH)
PHAHHAQAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 16 - protein, 101 aa, Artificial Sequence
  Rabbit IgG, Amino acid sequence for CH1 domain
GQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCS

SEQ ID NO: 17 - protein, 222 aa, Artificial Sequence
  Rabbit IgG, Amino acid sequence for <hinge>-CH2-CH3 portion (FcH)
KPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYNKLSVPTSEWQRGDVFTCSVMHEALHNHYTQKSISRSPGK

SEQ ID NO: 18 - protein, 103 aa, Artificial Sequence
  Murine IgG, Amino acid sequence for CL kappa domain
APTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 19 - protein, 103 aa, Artificial Sequence; identical to SEQ ID NO: 18
  Human IgG, Amino acid sequence for CL kappa domain

SEQ ID NO: 20 - protein, 100 aa, Artificial Sequence
  Rabbit IgG, Amino acid sequence for CL kappa domain
APTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

(19) United States
(12) Patent Application Publication
(10) Pub. No.: US 2021/0017272 A1
(43) Pub. Date: Jan. 21, 2021

(54) MULTIVALENT MONO- OR BISPECIFIC RECOMBINANT ANTIBODIES FOR ANALYTIC PURPOSE

(71) Applicant: Roche Diagnostics Operations, Inc., Indianapolis, IN (US)

(72) Inventors: Tobias Oelschlaegel, Muenchen (DE); Pavel Kubalec, Feldafing (DE)

(73) Assignee: Roche Diagnostics Operations, Inc., Indianapolis, IN (US)

(21) Appl. No.: 16/825,181

(22) Filed: Mar. 20, 2020

Related U.S. Application Data
(63) Continuation of application No. PCT/EP2018/075464, filed on Sep. 20, 2018.

(30) Foreign Application Priority Data
Sep. 22, 2017 (EP) 17192532.4

(51) Int. Cl. C07K 16/26; G01N 33/76; C07K 16/18; C07K 16/10; G01N 33/569; G01N 33/68

(52) U.S. Cl. CPC C07K 16/26; G01N 33/76; C07K 16/18; C07K 16/1045; G01N 33/56988; G01N 2333/4712; C07K 2317/31; C07K 2317/55; G01N 2333/59; G01N 2333/16; G01N 33/6887

(57) ABSTRACT

The present disclosure relates to novel analyte-specific multivalent recombinant antibodies that are particularly useful in immunoassays. Specifically, hexavalent, octavalent and decavalent antibodies are disclosed, including their construction, production, characterization and use in target antigen detection assays.

### CROSS-REFERENCE TO RELATED APPLICATIONS

This application is a continuation of International Application No. PCT/EP2018/075464 filed Sep. 20, 2018, which claims priority to European Application No. 17197532.4 filed Sep. 22, 2017, the disclosures of which are hereby incorporated by reference in their entirety.

The present disclosure relates to novel analyte-specific multivalent recombinant antibodies that are particularly useful in immunoassays. Specifically hexavalent, octavalent and decavalent antibodies are disclosed, their construction, production, characterization and use in target antigen detection assays.

### BACKGROUND OF THE INVENTION

An immunoassay is a biochemical test that measures the presence or concentration of a macromolecule or a small molecule in a solution, typically making use of an antibody as a specific detection agent. The molecule detected in the immunoassay is referred to as an “analyte” or “target analyte” and is in many cases a protein, although it may be other kinds of molecules, of different size and types, as long as the antibody or antibodies used in the assay are capable of facilitating specific detection of the analyte. In clinical diagnostics immunoassays frequently detect analytes in biological liquids such as serum, plasma or urine. More generally, as understood for the purpose of the present disclosure, immunoassays qualitatively or quantitatively detect an analyte in any kind of sample, provided that the sample is either a liquid sample or can be processed to become a liquid sample, and provided that the analyte to be detected is present as dissolved matter in aqueous solution being part of the liquid phase of the sample.

Immunoassays known to the art come in many different formats and variations. Immunoassays may be run in multiple steps with reagents being added and washed away or separated at different points in the assay. Multi-step assays are often called separation immunoassays or heterogeneous immunoassays. Some immunoassays can be carried out simply by mixing the reagents and sample and making a physical measurement. Such assays are called homogenous immunoassays or less frequently non-separation immunoassays.

Immunoassays rely on the ability of an antibody to recognize and specifically bind to the analyte even if the analyte is present in the sample as a minute quantity among a complex mixture of other molecules. The particular molecular structure recognized by an antibody is referred to as an “antigen” and the specific area on an antigen to which the antibody binds is called an “epitope”.

In addition to the specific binding of an antibody to the analyte, another key feature of all immunoassays is a means to produce a measurable signal in response to the binding. Frequently, an antibody is coupled with a detectable label. A large number of labels exist in modern immunoassays, and they allow for detection through different means. Many labels are detectable because they either emit radiation, produce a color change in a solution, fluoresce under light, or because they can be induced to emit light.

A typical embodiment of a heterogeneous immunoassay with antibodies comprises a so-called “sandwich” format, wherein two (a first and a second) distinct, non-overlapping antigens of the analyte are bound by a first and a second antibody, respectively. That is to say, by virtue of binding to the first and second antigens, the first and the second antibodies form a sandwich with the analyte. The first antibody is coupled to a detectable label; the second antibody is immobilized or capable of being immobilized, thereby allowing addition and removal of reagents as well as washing steps. A heterogeneous sandwich immunoassay comprises the generic steps of (a) contacting a sample containing the analyte with the first and the second antibody, wherein subsequently the analyte becomes bound (sandwiched) by the first and the second antibodies, wherein the second antibody is or becomes immobilized, followed by (b) detecting the amount of immobilized label being part of the sandwich. The amount of detected label corresponds to the amount of sandwiched analyte, and therefore corresponds to the amount of analyte in the sample.

In the technical field of preparing raw materials for immunoassays, antibody oligomers or polymers are known to the art; they are frequently used to enhance the antigen binding properties of the antibody. EP0955546A1 reports a chemically polymerized antibody conjugate which is labeled with a dye. The antibody polymerization product is characterized by a larger number of functional antigen binding sites, i.e. it is “multivalent” binder. When used in an immunoassay the multivalent binder reportedly results in an improvement of antigen binding sensitivity. The polymerized antibody bound to a detectable label is described for use in antigen-antibody reactions for diagnostic purposes.

Using an enzyme as a detectable label, EP175560A2 reports a process for making a polymeric enzyme/antibody conjugate by covalently coupling a pre-polymerized enzyme to an antibody or fragment thereof, such as a Fab, Fab′ or F(ab′)2 fragment. The document further discloses an immunoassay for determination of an analyte in a liquid sample which comprises the steps of (a) forming a complex of the conjugate with the analyte, (b) separating the complex, (c) detecting enzymatic activity in the complex, and (d) relating the detected enzymatic activity to the amount of analyte in the sample. The document further mentions optimization of the production of the conjugate with respect to the stoichiometry of antibody or fragments thereof on the one hand, and the pre-polymerized enzyme on the other hand. The stoichiometry reportedly has an impact on detection sensitivity and background activity in the immunoassay as disclosed.

US20030143638A1 reports a method for adjusting the reactivity of an antigen-antibody reaction. This method comprises steps of (a) obtaining a plurality of antibody polymers having different degrees of polymerization; (b) bonding the plurality of antibody polymers to carriers thereby obtaining a set of antibody/carrier complexes; and (c) selecting an antibody/carrier complex which reacts with an antigen at a desired degree of reaction, from the set of antibody/carrier complexes.

Each arm of an antibody that binds to the target antigen is referred to as the antigen-binding (=Fab) fragment. Fab designates “fragment antigen-binding”; a region on an antibody that binds to antigens composed of one constant and one variable domain of each of the heavy (Fab_{H}) and light chains (Fab_{L}). A Fab fragment thus comprises two aligned polypeptides, a first fragment of the heavy chain (Fab_{H}) and the unfragmented light chain (Fab_{L}) which is aligned with the heavy chain fragment and connected via a disulfide bridge. The tail region of an antibody is usually called fragment crystalizable (=Fc) region and comprises in IgG, IgA and IgD antibody isotypes two further fragments of the heavy chain (each one referred to as Fc_{H}) which are identical and aligned with each other and which are connected with one or more disulfide bridges. Proteolytic processing of an immunoglobulin of IgG, IgA or IgD isotype can be used to artificially cleave an antibody to generate Fc and Fab fragments which can be separated and isolated. The enzyme papain can be used to cleave a single immunoglobulin into two Fab fragments and one Fc fragment. The enzyme pepsin cleaves below the hinge region, thereby yielding a F(ab′)2 fragment and a pFc′ fragment. Similarly, the enzyme IdeS specifically cleaves at the hinge region of IgG.

Antigen-binding antibody fragments without Fc portions (obtained in isolated form following proteolytic processing e.g. with pepsin or papain) are particularly preferred if the sample with the target analyte is whole blood, blood serum or blood plasma. It is known that components contained in such samples can bind unspecifically to the Fc portions of conventional antibodies. Unspecific interaction of sample components with Fc portions can increase background signal of an immunoassay. Increased background signal has a negative impact on assay performance as the signal-to-noise ratio is decreased.

A specific embodiment of the prior art involves the step of chemically cross-linking antigen-binding antibody fragments after removal of their Fc portions, thereby forming a polymer of the fragments. Such polymerized antigen-binding antibody fragments are presently preferred in a number of immunoassays that require high sensitivity for the target antigen and low background signal. The crosslinking step is performed with the intention to generate a multivalent analyte-specific binder with enhanced binding properties. For a number of commercially available immunoassays, an antibody-derived multivalent analyte-specific binder without an Fc portion and bound to a label is a preferred detection agent known to the art. In certain cases, chemically crosslinked (i.e. polymerized) antibody fragments are advantageous over antibodies in their naturally occurring form which still include the Fc portions, in that signal-to-noise ratio of the immunoassay can be improved by this means.

Naturally occurring, unfragmented antibodies comprise two heavy chains that are linked together by disulfide bonds and two light chains. Each single light chain is linked to one of the heavy chains by disulfide bonds. Each Fab_{H} portion within an immunoglobulin heavy chain has at the N-terminal end a variable domain (VH) followed by a number of constant domains (three or four constant domains, CH1, CH2, CH3 and CH4, depending on the antibody class). Each Fab_{L} light chain has a variable domain (VL) at the N-terminal end and a constant domain (CL) at its other (C-terminal) end; the constant domain of the light chain is aligned with the first constant domain (CH1) of the heavy chain, and the light chain variable domain (VL) is aligned with the variable domain of the heavy chain (VH). Particular amino acid residues are believed to form an interface between the light and heavy chain domains which mediates the alignment by physicochemical interactions.

The constant domains are not involved directly in binding of the antibody to its target antigen, but they are involved in various effector functions in vivo. The variable domains of each pair of light and heavy chains are involved directly in the binding of the antibody to its epitope. The variable domains of naturally occurring light (VL) and heavy (VH) chains have the same general structure; each comprises four framework regions (FRs), whose sequences are somewhat conserved, connected by three complementarity determining regions (CDRs). The CDRs in each chain are held in close proximity by the FRs; the epitope binding site is formed by the combined CDRs of the aligned light and the heavy chain in the respective Fab portion of the antibody.

A variety of recombinant multispecific antibody formats have been developed in the recent past, e.g. tetravalent bispecific antibodies by fusion of, e.g. an IgG antibody format and single chain domains (see e.g. Coloma, M. J., et. al., Nature Biotech. 15 (1997) 159-163; WO 2001/077342; and Morrison, S. L., Nature Biotech. 25 (2007) 1233-1234). Also several other new formats, wherein the antibody core structure (IgA, IgD, IgE, IgG or IgM) is no longer retained, have been developed; such as dia-, tria- or tetrabodies, minibodies and several single chain formats (scFv, Bis-scFv). A number of these are capable of binding two or more antigens (Holliger, P., et. al, Nature Biotech. 23 (2005) 1126-1136; Fischer, N., and Léger, O., Pathobiology 74 (2007) 3-14; Shen, J., et. al., J. Immunol. Methods 318 (2007) 65-74; Wu, C., et al., Nature Biotech. 25 (2007) 1290-1297). Such formats use linkers either to fuse the antibody core (IgA, IgD, IgE, IgG or IgM) to a further binding protein (e.g. scFv) or to fuse e.g. two Fab fragments or scFv (Fischer, N., and Léger, O., Pathobiology 74 (2007) 3-14).

WO 2001/077342 discloses different engineered antibodies. A particular engineered antibody of the IgG class is disclosed which comprises four antigen binding sites. Specifically, the N-terminal CH1-VH portion of each heavy chain is extended with a further CH1-VH portion. Accordingly, in the fully assembled antibody the N-terminal portion of each heavy chain is aligned and linked not with one but with two corresponding light chains. Each arm of such a tetravalent antibody thus comprises a first and a second antigen binding site, the two sites being arranged in tandem. The document mentions that such engineered antibodies may be useful in diagnostic assays, e.g. in detecting antigens of interest in specific cells, tissues or serum.

Additional overview on the topic of engineered antibodies was provided by Chiu M. L. & Gillilang G. L. Curr Opin Struct Biol 38 (2016) 163-173 and Tiller K. L. & Tessier P. M. Annu Rev Biomed Eng 17 (2015) 191-216. A number of different multivalent antibody architectures were discussed by Deyev S. M. & Lebedenko E. N. (BioEssays 30 (2008) 904-918).

Hexavalent engineered antibodies are disclosed by Blanco-Toribio A. et al. (mAbs 5 (2013) 70-79). A bispecific decavalent antibody was reported by Stone E. et al. (J Immunol Methods 318 (2007) 88-94). The above illustrates a desire in the art to provide modified immunoglobulins as raw materials for immunoassays that have reduced background signal. Further, immunoglobulins are desired which allow for a high sensitivity of the assay regarding the target analyte to be detected.

Generation of multivalent antigen-binding macromolecules by way of chemically linking or cross-linking (polymerizing) whole antibodies or antibody fragments is an already practiced approach to address these technical objectives. But formation of chemical linkages is a stochastic process, in several aspects. To begin with, chemical cross-linking is not (or only to a limited extent) site-specific. That is to say, there are always several accessible amino acid side chains in a polypeptide which in principle can be reacted in a cross-linking reaction. And for each side chain to be considered there is a different probability whether or not it takes part in a reaction. The chemical reaction can be quantitative or non-quantitative. Further, as a matter of principle, the chemical reaction on the antibodies or antibody fragments does not lead to a single product but yields a range of different products. The implication is that there can be no prediction as to which particular amino acid side chains of a first and a second polypeptide are cross-linked.

Also, there is typically a distribution regarding the average number of polypeptides which are connected to form a multivalent antigen-binding macromolecule. Chemical cross-linking reactions lead to a distribution of molecular weights of the products, reflecting the number of antibodies or antibody fragments that are connected. Usually, however, only a fraction of the products is of actual use and technically suited as multivalent antigen-binding macromolecules in an immunoassay. Therefore, in order to come up with a sufficiently standardized and reproducible assay, a desired subfraction of the products has to be identified, separated, purified and characterized for further use.

Accordingly, there is a need in the art for improved provision of multivalent antigen-binding macromolecules suitable for use as detection reagents in immunoassays. Such macromolecules are desired to be biochemically stable and designed such that there is convenience in the construction process. Further, recombinant constructs for multivalent antigen-binding macromolecules are desired which can be expressed in transformed host cells, wherein the chance of success is high concerning expression and production of the desired product in good quantities. The basis of the present disclosure is the surprising finding that multivalent recombinant antibodies as reported herein can advantageously produced recombinantly at high quantities in stably transformed cells. Expression levels have been observed which are comparable to recombinantly expressed conventional immunoglobulins. The multivalent recombinant antibodies reported herein are of great advantage when used in a diagnostic assay for detecting an analyte. In this respect, the reported recombinant antibodies improve the signal-to-noise ratio of immunoassays, particularly when compared with conventional immunoglobulins.

### SUMMARY OF THE INVENTION

As a first aspect related to all other aspects and embodiments reported herein, the present disclosure provides a multivalent recombinant antibody, wherein the antibody comprises a number of p light chain polypeptides Fab_{L} and a dimer of two heavy chain polypeptides, wherein each heavy chain polypeptide has a structure of Formula I

_{N-terminus}[Fab_{H}-L-]ₙFab_{H}-L-dd(Fc_{H})[-L-Fab_{H}]_{m C-terminus} (Formula I)

wherein
(a) p is a value selected from the group consisting of 6, 8, and 10,
each of m and n is selected independently from an integer of 1 to 3, and
each of m and n is selected such that the value of p equals (2+2*(n+m));
(b) “-” is a covalent bond within a polypeptide chain;
(c) each L is optional and, if present, is an independently selected variable linker amino acid sequence;
(d) each dd(Fc_{H}) is a heavy chain dimerization region of a heavy chain of a non-antigen binding immunoglobulin region;
(e) in the dimer the two dd(Fc_{H}) are aligned with each other in physical proximity;
(f) each Fab_{H} is independently selected from A_{H} and B_{H}, wherein A_{H} and B_{H} are different, and A_{H} and B_{H} are independently selected from the group consisting of

_{N-terminus}[VH-CH1]_{H C-terminus} (Formula II),

_{N-terminus}[VH-CL]_{H C-terminus} (Formula III),

_{N-terminus}[VL-CL]_{H C-terminus} (Formula IV), and

_{N-terminus}[VL-CH1]_{H C-terminus} (Formula V),
wherein
VH is a N-terminal immunoglobulin heavy chain variable domain,
VL is a N-terminal immunoglobulin light chain variable domain,
CH1 is a C-terminal immunoglobulin heavy chain constant domain 1, and
CL is a C-terminal immunoglobulin light chain constant domain;
(g) each Fab_{L} is independently selected from A_{L} and B_{L}, wherein A_{L} and B_{L} are different, and A_{L} and B_{L} are independently selected from the group consisting of

_{N-terminus}[VH-CH1]_{L C-terminus} (Formula VI),

_{N-terminus}[VH-CL]_{L C-terminus} (Formula VII),

_{N-terminus}[VL-CL]_{L C-terminus} (Formula VIII), and

_{N-terminus}[VL-CH1]_{L C-terminus} (Formula IX);
(h) each antigen binding site Fab_{H}:Fab_{L} of the antibody is an aligned pair (the alignment being signified by “:”), wherein each aligned pair is independently selected from the group consisting of A_{H}:A_{L} and B_{H}:B_{L}, wherein A_{H}:A_{L} and B_{H}:B_{L} are selected independently from the group consisting of

[VL-CH1]_{H}:[VH-CL]_{L},

[VL-CL]_{H}:[VH-CH1]_{L},

[VH-CH1]_{H}:[VL-CL]_{L}, and

[VH-CL]_{H}:[VL-CH1]_{L},

and wherein in each aligned pair the respective CL and CH1 are covalently linked via a disulfide bond.

As a second aspect related to all other aspects and embodiments reported herein, the present disclosure provides the use of a multivalent antibody as disclosed herein in an assay for the detection of an antigen. As a third aspect related to all other aspects and embodiments reported herein, the present disclosure provides a kit comprising a chimeric or non-chimeric multivalent recombinant antibody as disclosed in the first aspect of the present disclosure.

A fourth aspect related to all other aspects and embodiments reported herein, the present disclosure provides a method for detecting an antigen, the method comprising the steps of contacting a multivalent recombinant antibody as disclosed in the first aspect of the present disclosure with the antigen, thereby forming a complex of antigen and multivalent recombinant antibody, followed by detecting formed complex, thereby detecting the antigen. In a specific embodiment, the method comprises the steps of (a) mixing a multivalent recombinant antibody according to the present disclosure with a liquid sample suspected of containing the antigen, (b) incubating the sample and the multivalent recombinant antibody of step (a), thereby forming a complex of antigen and multivalent recombinant antibody if antigen is present and accessible for contact with the multivalent recombinant antibody during the incubation, (c) detecting complex formed in step (b), thereby detecting the antigen.

### DESCRIPTION OF THE FIGURES

FIG. 1A schematically depicts an oligomer of antibody fragments chemically linked to each other.

FIG. 1B shows a SEC chromatograph representing the outcome of an exemplary cross-linking experiment using F(ab′)2 fragments to generate oligomers. For further details see Example 2.

FIG. 1C shows steps in manufacturing antibody fragments useful in the present method.

FIG. 2A schematically depicts an oligomer of antibody fragments chemically linked to each other.

FIG. 2B shows a bivalent monoclonal antibody of IgG isotype.

FIG. 2C shows a multivalent antibody with four antigen binding sites.

FIG. 2D shows a multivalent antibody with six antigen binding sites.

FIG. 2E shows a multivalent antibody with eight antigen binding sites.

FIG. 2F shows a multivalent antibody with twelve antigen binding sites.

FIG. 3A shows a map of an exemplary expression vector for the heavy chain of a multivalent antibody.

FIG. 3B shows a map of an exemplary expression vector for a light chain. For further details see Example 3.

FIG. 4A shows SEC chromatograms. For further details see Examples 4 and 5.

FIG. 4B shows SEC chromatograms. For further details see Examples 4 and 5.

FIGS. 4C and 4D show SEC chromatograms. For further details see Examples 4 and 5.

FIGS. 5A and 5B show SEC chromatograms. For further details see Example 5.

FIG. 6 shows a PAGE electropherogram. For further details see Example 5.

FIGS. 7A-7F show normalized signal-to-noise values of labeled antibodies. For further details see Example 9.

FIG. 8A and FIG. 8B show SEC chromatograms for a standard and a TN-T multivalent antibody as disclosed in Example 11.

FIG. 9A and FIG. 9B show SEC chromatograms for a standard and a multivalent antibody against the HIV p24 antigen as disclosed in Example 12, monospecific 2E7.

FIG. 9C and FIG. 9D show SEC chromatograms for a standard and a multivalent antibody against the HIV p24 antigen as disclosed in Example 12, monospecific 2E7.

FIG. 10A and FIG. 10B show SEC chromatograms for a standard and a multivalent antibody against the HIV p24 antigen as disclosed in Example 12, monospecific 6D9.

FIG. 10C shows an SEC chromatogram for a standard and a multivalent antibody against the HIV p24 antigen as disclosed in Example 12, monospecific 6D9.

FIG. 11A and FIG. 11B show SEC chromatograms for a standard and a multivalent antibody against the HIV p24 antigen as disclosed in Example 12, A size marker, B monospecific 6D9, C bispecific 6D9/2E7.

FIG. 11C shows SEC chromatograms for a standard and a multivalent antibody against the HIV p24 antigen as disclosed in Example 12, A size marker, B monospecific 6D9, C bispecific 6D9/2E7.

FIG. 12A and FIG. 12B show Electrochemiluminescence signal counts generated by Ruthenium-labeled antibodies. For further details see Example 8.

FIG. 13A and FIG. 13B show SEC chromatograms for a standard and a TN-T multivalent antibody as disclosed in Example 11.

FIG. 14A and FIG. 14B show SEC chromatograms for a standard and multivalent antibodies against the HIV p24 antigen as disclosed in Example 12.

FIG. 14C and FIG. 14D show SEC chromatograms for a standard and multivalent antibodies against the HIV p24 antigen as disclosed in Example 12.

FIG. 14E and FIG. 14F show SEC chromatograms for a standard and multivalent antibodies against the HIV p24 antigen as disclosed in Example 12.

FIG. 14G and FIG. 14H show SEC chromatograms for a standard and multivalent antibodies against the HIV p24 antigen as disclosed in Example 12.

FIGS. 14I and 14J show SEC chromatograms for a standard and multivalent antibodies against the HIV p24 antigen as disclosed in Example 12.

### DETAILED DESCRIPTION OF THE INVENTION

The terms “a”, “an” and “the” generally include plural referents, unless the context clearly indicates otherwise.

The term “antibody” herein is used in the broadest sense and encompasses various antibody structures, including but not limited to structures known from monoclonal antibodies, polyclonal antibodies, multispecific antibodies (e.g., bispecific antibodies), and antibody fragments so long as they exhibit the desired antigen-binding activity.

The term “antibody specificity” refers to selective recognition of a particular epitope of an antigen by the antibody. Natural antibodies, for example, are monospecific. The term “monospecific antibody” as used herein denotes an antibody that has one or more binding sites each of which bind to the same epitope of the same antigen. Thus, “monospecific” antibodies are antibodies that bind to a single epitope. By way of non-limiting example, monoclonal antibodies are monospecific. In more general terms, an antibody capable of binding only a single epitope is understood to be monospecific. It is understood for the purpose of the present disclosure and all aspects and embodiments reported herein that more than one binding site may exist or can be found with respect to a single epitope, wherein the binding sites are specific for the epitope. Thus, the term “monospecific” encompasses different binding sites as long as these can be commonly defined by their specificity against the same epitope. In this regard, a monospecific antibody may encompass binding sites which differ by their respective kinetics of epitope binding.

“Bispecific”, “trispecific”, “tetraspecific”, “pentaspecific”, “hexaspecific”, etc. antibodies, also referred to as “multispecific” antibodies bind two or more different epitopes (for example, two, three, four, or more different epitopes). The epitopes may be identical or non-identical, and they may be on the same or on different antigens. An example of a multispecific antibody is a “bispecific antibody” which binds two different epitopes. Generally, when an antibody possesses more than one single specificity, the recognized epitopes may be associated with a single antigen or with more than one antigen.

The term “valent” as used herein denotes the presence of a specified number of binding sites in an antibody molecule. A natural antibody of the IgG class of immunoglobulins for example has two binding sites and therefore is bivalent. As such, the term “trivalent” denotes the presence of three binding sites in an antibody molecule, “tetravalent” denotes four binding sites, “hexavalent” denotes six binding sites, and so forth.

“Conservative substitutions” applies to both amino acid and nucleic acid sequences. With respect to particular nucleic acid sequences, “conservatively substituted” refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of ordinary skill in the art will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of ordinary skill in the art will recognize that individual substitutions in a peptide, polypeptide, or protein sequence which alter a single amino acid or a small percentage of amino acids in the amino acid sequence is a “conservative substitution” where the alteration results in the substitution of an amino acid with a chemically similar amino acid. Conservative substitution tables providing functionally similar amino acids are known to those of ordinary skill in the art. Conservative substitution tables providing functionally similar amino acids are known to those of ordinary skill in the art. The following eight groups each contain amino acids that are conservative substitutions for one another:

The term “conservative amino acid substitutions” refers to all substitutions wherein the substituted amino acid has similar structural or chemical properties with the corresponding amino acid in the reference sequence. By way of example, conservative amino acid substitutions involve substitution of one aliphatic or hydrophobic amino acids, e.g., alanine, valine, leucine, isoleucine, methionine, phenylalanine, or tryptophan with another; substitution of one hydroxyl-containing amino acid, e.g., serine and threonine, with another; substitution of one acidic residue, e.g., glutamic acid or aspartic acid, with another; replacement of one amide-containing residue, e.g., asparagine and glutamine, with another; replacement of one aromatic residue, e.g., phenylalanine and tyrosine, with another; replacement of one basic residue, e.g., lysine, arginine and histidine, with another; and replacement of one small amino acid, e.g., alanine, serine, threonine, and glycine, with another.

As used herein “deletions” and “additions” in reference to amino acid sequence, means deletion or addition of one or more amino acids to the amino terminus, the carboxy-terminus, the interior of the amino acid sequence or a combination thereof, for example the addition can be to one of the antibodies subject of the present application.

As used herein, “homologous sequences” have amino acid sequences which are at least 70%, at least 80%, at least 90%, at least 95%, or at least 99% homologous to the corresponding reference sequences. Sequences which are at least 90% identical have no more than 1 alteration, i.e., any combination of deletions, additions or substitutions, per 10 amino acids of the reference sequence. Percent homology is determined by comparing the amino acid sequence of the variant with the reference sequence using, for example, MEGALIGN™ project in the DNA STAR™ program.

The terms “identical” or percent “identity,” in the context of two or more nucleic acids or polypeptide sequences, refer to two or more sequences or subsequences that are the same. Sequences are “substantially identical” if they have a percentage of amino acid residues or nucleotides that are the same (i.e., about 60% identity, about 65%, about 70%, about 75%, about 80%, about 85%, about 90%, or about 95% identity over a specified region), when compared and aligned for maximum correspondence over a comparison window, or designated region as measured using one of the following sequence comparison algorithms (or other algorithms available to persons of ordinary skill in the art) or by manual alignment and visual inspection. This definition also refers to the complement of a test sequence. The identity can exist over a region that is at least about 50 amino acids or nucleotides in length, or over a region that is 75-100 amino acids or nucleotides in length, or, where not specified, across the entire sequence of a polynucleotide or polypeptide. A polynucleotide encoding a polypeptide of the present disclosure, including homologs from species other than human, may be obtained by a process comprising the steps of screening a library under stringent hybridization conditions with a labeled probe having a polynucleotide sequence of the present disclosure or a fragment thereof, and isolating full-length cDNA and genomic clones containing said polynucleotide sequence. Such hybridization techniques are well known to the skilled artisan.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

“Antibody fragments” comprise a portion of a full length antibody, preferably the variable domain thereof, or at least the antigen binding site thereof. Examples of antibody fragments include diabodies, single-chain antibody molecules, and multispecific antibodies formed from antibody fragments. scFv antibodies are, e.g., described in Huston, J. S., Methods in Enzymol. 203 (1991) 46-88. In addition, antibody fragments comprise single chain polypeptides having the characteristics of

a V_{H} domain, namely being able to assemble together with a V_{L} domain, or of a V_{L} domain binding to IGF-1, namely being able to assemble together with a V_{H} domain to a functional antigen binding site and thereby providing the properties of an antibody according to the invention.

The terms “monoclonal antibody” or “monoclonal antibody composition” as used herein refer to a preparation of antibody molecules of a single amino acid composition.

The term “specific binding agent” is used to indicate that an agent is used which is able to either specifically bind to or to be specifically bound by an analyte of interest. Many different assay set-ups for immunoassays are known in the art. Dependent on the specific assay set-up, various biotinylated specific binding agents can be used. In one embodiment the biotinylated specific binding agent is selected from the group consisting of a biotinylated analyte-specific binding agent, a biotinylated analyte bound to solid phase, and a biotinylated antigen bound to solid phase.

The term “analyte-specific binding agent” refers to a molecule specifically binding to the analyte of interest. An analyte-specific binding agent in the sense of the present disclosure typically comprises binding or capture molecules capable of binding to an analyte (other terms analyte of interest; target molecule). In one embodiment the analyte-specific binding agent has at least an affinity of 10⁷ l/mol for its corresponding target molecule, i.e. the analyte. The analyte-specific binding agent in other embodiments has an affinity of 10⁸ l/mol or even of 10⁹ l/mol for its target molecule. As the skilled artisan will appreciate the term specific is used to indicate that other biomolecules present in the sample do not significantly bind to the binding agent specific for the analyte. In some embodiments, the level of binding to a biomolecule other than the target molecule results in a binding affinity which is only 10%, more preferably only 5% of the affinity of the target molecule or less. In one embodiment no binding affinity to other molecules than to the analyte is measurable. In one embodiment the analyte-specific binding agent will fulfill both the above minimum criteria for affinity as well as for specificity.

The term “analyte-specific binding” as used in the context of an antibody refers to the immunospecific interaction of the antibody with its target epitope on the analyte, i.e. the binding of the antibody to the epitope on the analyte. The concept of analyte-specific binding of an antibody via its epitope on an analyte is fully clear to the person skilled in the art.

The terms “polypeptide,” “peptide” and “protein” refer to a polymer of amino acid residues. The terms apply to naturally occurring amino acid polymers as well as amino acid polymers in which one or more amino acid residues are a non-naturally encoded amino acid. As used herein, the terms encompass amino acid chains, wherein the amino acid residues are linked by covalent peptide bonds. The polypeptides, peptides and proteins are written using standard sequence notation, with the nitrogen terminus being on the left and the carboxy terminus on the right. Standard single letter notations have been used as follows: A—alanine, C—cysteine, D—aspartic acid, E—glutamic acid, F—phenylalanine, G—glycine, H—histidine, S—Isoleucine, K—lysine, L—leucine, M—methionine, N—asparagine, P—proline, Q—glutamine, R—arginine, S—serine, T—threonine, V—valine, W—tryptophan, Y—tyrosine. The term “peptide” as used herein refers to a polymer of amino acids that has a length of up to 5 amino acids. The term “polypeptide” as used herein refers to a polymer of amino acids that has a length of 6 or more amino acids. The term “protein” either signifies a polypeptide chain or a polypeptide chain with further modifications such as glycosylation, phosphorylation, acetylation or other post-translational modifications

“Antibody fragments” comprise a portion of an intact antibody, preferably comprising the antigen-binding region thereof. Examples of antibody fragments include Fab, Fab′, F(ab′)₂, and Fv fragments; single-chain antibody molecules; scFv, sc(Fv)2; diabodies; and multispecific antibodies formed from antibody fragments.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen.

The Fab fragment contains the heavy- and light-chain variable domains and also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody-hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The term “monoclonal antibody” as used herein refers to an antibody obtained from a population of substantially homogeneous antibodies, i.e., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target-binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations, which typically include different antibodies directed against different determinants (epitopes), each monoclonal antibody of a monoclonal-antibody preparation is directed against a single determinant on an antigen. In addition to their specificity, monoclonal-antibody preparations are advantageous in that they are typically uncontaminated by other immunoglobulins.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler and Milstein., Nature, 256:495-97 (1975); Hongo et al., Hybridoma, 14 (3): 253-260 (1995), Harlow et al., Antibodies: A Laboratory Manual, (Cold Spring Harbor Laboratory Press, 2^{nd} ed. 1988); Haemmerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, PNAS USA 101(34): 12467-12472 (2004); and Lee et al., J. Immunol. Methods 284(1-2): 119-132(2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., PNAS USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and U.S. Pat. No. 5,661,016; Marks et al., Bio/Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg and Huszar, Intern. Rev. Immunol. 13: 65-93 (1995).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (e.g., U.S. Pat. No. 4,816,567 and Morrison et al., PNAS USA 81:6851-6855 (1984)). Chimeric antibodies include PRIMATIZED® antibodies wherein the antigen-binding region of the antibody is derived from an antibody produced by, e.g., immunizing macaque monkeys with the antigen of interest.

The term “hypervariable region,” “HVR,” or “HV,” when used herein refers to the regions of an antibody-variable domain which are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al. Immunity 13:37-45 (2000); Johnson and Wu in Methods in Molecular Biology 248:1-25 (Lo, ed., Human Press, Totowa, N.J., 2003). Indeed, naturally occurring camelid antibodies consisting of a heavy chain only are functional and stable in the absence of light chain. See, e.g., Hamers-Casterman et al., Nature 363:446-448 (1993) and Sheriff et al., Nature Struct. Biol. 3:733-736 (1996).

A number of HVR delineations are in use and are encompassed herein. The HVRs that are Kabat complementarity-determining regions (CDRs) are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk J. Mol. Biol. 196:901-917 (1987)). The AbM HVRs represent a compromise between the Kabat CDRs and Chothia structural loops, and are used by Oxford Molecular's AbM antibody-modeling software. The “contact” HVRs are based on an analysis of the available complex crystal structures. The residues from each of these HVRs are noted below.

| Loop Kabat | AbM | Chothia | Contact |
|---|---|---|---|
| L1 L24-L34 | L24-L34 | L26-L32 | L30-L36 |
| L2 L50-L56 | L50-L56 | L50-L52 | L46-L55 |
| L3 L89-L97 | L89-L97 | L91-L96 | L89-L96 |
| H1 H31-H35B | H26-H35B | H26-H32 | H30-H35B (Kabat Numbering) |
| H1 H31-H35 | H26-H35 | H26-H32 | H30-H35 (Chothia Numbering) |
| H2 H50-H65 | H50-H58 | H53-H55 | H47-H58 |
| H3 H95-H102 | H95-H102 | H96-H101 | H93-H101 |

HVRs may comprise “extended HVRs” as follows: 24-36 or 24-34 (L1), 46-56 or 50-56 (L2), and 89-97 or 89-96 (L3) in the VL, and 26-35 (H1), 50-65 or 49-65 (H2), and 93-102, 94-102, or 95-102 (H3) in the VH.

The variable-domain residues are numbered according to Kabat et al., supra, for each of these extended-HVR definitions.

The expression “variable-domain residue-numbering as in Kabat” or “amino-acid-position numbering as in Kabat,” and variations thereof, refers to the numbering system used for heavy-chain variable domains or light-chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy-chain variable domain may include a single amino acid insert (residue 52a according to Kabat) after residue 52 of H2 and inserted residues (e.g. residues 82a, 82b, and 82c, etc. according to Kabat) after heavy-chain FR residue 82. The Kabat numbering of residues may be determined for a given antibody by alignment at regions of homology of the sequence of the antibody with a “standard” Kabat numbered sequence.

The term “experimental animal” denotes a non-human animal. In one embodiment the experimental animal is selected from rat, mouse, hamster, rabbit, camel, llama, non-human primates, sheep, dog, cow, chicken, amphibians, sharks and reptiles. In one embodiment the experimental animal is a rabbit.

An epitope is a region of an antigen that is bound by a binding site of an antibody. The term “epitope” includes any determinant capable of specific binding to an antibody. In certain embodiments, epitope determinants include chemically active surface groupings of molecules such as amino acids, glycan side chains, phosphoryl, or sulfonyl, and, in certain embodiments, may have specific three dimensional structural characteristics, and/or specific charge characteristics.

As used herein, the terms “binding” and “specific binding” refer to the binding of the antibody to an epitope of the antigen in an in vitro assay, particularly in a plasmon resonance assay (BIAcore, GE-Healthcare Uppsala, Sweden) with purified antigen. In certain embodiments, an antibody is said to specifically bind an antigen when it preferentially recognizes its target antigen in a complex mixture of proteins and/or macromolecules.

The affinity of the binding of an antibody to an antigen is defined by the terms kₐ (rate constant for the association of the antibody from the antibody/antigen complex), k_{d} (dissociation rate constant), and K_{D} (k_{d}/kₐ). In one embodiment binding or that/which specifically binds to means a binding affinity (K_{D}) of 10⁻⁷ mol/l or less, in one embodiment 10⁻⁷ M to 10⁻¹³ mol/l. Thus, a multispecific antibody in all aspects and embodiments disclosed herein specifically binds to each target antigen for which it is specific with a binding affinity (K_{D}) of 10⁻⁷ mol/l or less, e.g. with a binding affinity (K_{D}) of 10⁻⁸ to 10⁻¹³ mol/l. In one embodiment with a binding affinity (K_{D}) of 10⁻⁸ to 10⁻¹³ mol/l. In this regard, the target antigen can be a single molecule or different molecules. In a specific embodiment the target antigen is a complex formed by two or more different molecules, wherein the multispecific antibody specifically binds to the complex with a binding affinity (K_{D}) of 10⁻⁷ mol/l or less.

The terms “monoclonal antibody” or “monoclonal antibody composition” as used herein refer to a preparation of antibody molecules of a single amino acid composition. The recombinant antibody according to all aspects and embodiments disclosed herein as understood to be encompassed by the term “monoclonal antibody”.

The term “chimeric” antibody refers to an antibody in which a portion of the heavy and/or light chain is derived from a particular source or species, while the remainder of the heavy and/or light chain is derived from a different source or species. In a specific embodiment, the recombinant antibody according to all aspects and embodiments disclosed herein may contain a Fab_{H}:Fab_{L} portion originating in a first species and an Fc_{H} portion of a second species. In another specific embodiment, the recombinant antibody comprises a plurality of specificities with different Fab_{H}:Fab_{L} portions derived from different sources or species.

The terms “binding site” or “antigen-binding site” as used herein denote the region(s) of an antibody molecule to which a ligand (e.g. the antigen or antigen fragment of it) actually binds and which is derived from an antibody. The antigen-binding site includes antibody heavy chain variable domains (VH) and/or antibody light chain variable domains (VL), or pairs of VH/VL.

The antigen-binding sites that specifically bind to the desired antigen can be derived a) from known antibodies specifically binding to the respective target antigen or b) from new antibodies or antibody fragments obtained by de novo immunization methods using inter alia either the antigen protein or nucleic acid encoding a protein as target antigen or fragments thereof or by phage display.

An antigen-binding site of an antibody including a recombinant antibody as disclosed in all aspects and embodiments herein can contain six complementarity determining regions (CDRs) which contribute in varying degrees to the affinity of the binding site for the epitope of the antigen. There are three heavy chain variable domain CDRs (CDRH1, CDRH2 and CDRH3) and three light chain variable domain CDRs (CDRL1, CDRL2 and CDRL3). The extent of CDR and framework regions (FRs) is determined by comparison to a compiled database of amino acid sequences in which those regions have been defined according to variability among the sequences. Also included within the scope of the invention are functional antigen binding sites comprised of fewer CDRs (i.e., where binding specificity is determined by three, four or five CDRs). For example, less than a complete set of 6 CDRs may be sufficient for binding. In some cases, a VH or a VL domain will be sufficient.

The “light chains” of antibodies (immunoglobulins) from any vertebrate species can be assigned to one of two distinct types, called kappa (κ) and lambda (λ), based on the amino acid sequences of their constant domains. A wild type light chain typically contains two immunoglobulin domains, usually one variable domain (VL) that is important for binding to an antigen and a constant domain (CL).

Several different types of “heavy chains” exist that define the class or isotype of an antibody. A wild type heavy chain contains a series of immunoglobulin domains, usually with one variable domain (VH) that is important for binding antigen and several constant domains (CH1, CH2, CH3, etc.).

The term “Fc domain” is used herein to define a C-terminal region of an immunoglobulin heavy chain that contains at least a portion of the constant region. For example in natural antibodies, the Fc domain is composed of two identical protein fragments, derived from the second and third constant domains of the antibody's two heavy chains in IgG, IgA and IgD isotypes; IgM and IgE Fc domains contain three heavy chain constant domains (CH domains 2-4) in each polypeptide chain. “Devoid of the Fc domain” as used herein means that the bispecific antibodies of the invention do not comprise a CH2, CH3 and CH4 domain; i.e. the constant heavy chain consists solely of one or more CH1 domains.

The “variable domains” or “variable region” as used herein denotes each of the pair of light and heavy chains which is involved directly in binding the antibody to the antigen. The variable domain of a light chain is abbreviated as “VL” and the variable domain of a light chain is abbreviated as “VH”. The variable domains of human light chains and heavy chains have the same general structure. Each variable domain comprises four framework (FR) regions, the sequences of which are widely conserved. The FR are connected by three “hypervariable regions” (or “complementarity determining regions”, CDRs). CDRs on each chain are separated by such framework amino acids. Therefore, the light and heavy chains of an antibody comprise from N- to C-terminal direction the domains FR1, CDR1, FR2, CDR2, FR3, CDR3, and FR4. The FR adopt a β-sheet conformation and the CDRs may form loops connecting the β-sheet structure. The CDRs in each chain are held in their three-dimensional structure by the FR and form together with the CDRs from the other chain an “antigen binding site”. Especially, CDR3 of the heavy chain is the region which contributes most to antigen binding. CDR and FR regions are determined according to the standard definition of Kabat, et al., Sequences of Proteins of Immunological Interest, 5th ed., Public Health Service, National Institutes of Health, Bethesda, Md. (1991).

The term “constant domains” or “constant region” as used within the current application denotes the sum of the domains of an antibody other than the variable region. The constant region is not directly involved in binding of an antigen, but exhibits various effector functions.

Depending on the amino acid sequence of the constant region of their heavy chains, antibodies are divided in the classes: IgA, IgD, IgE, IgG and IgM, and several of these may are further divided into subclasses, such as IgG1, IgG2, IgG3, and IgG4, IgA1 and IgA2. The heavy chain constant regions that correspond to the different classes of antibodies are called are called α, δ, ε, γ and μ, respectively. The light chain constant regions (CL) which can be found in all five antibody classes are called κ (kappa) and λ (lambda). The “constant domains” as used herein are from human origin, which is from a constant heavy chain region of a human antibody of the subclass IgG1, IgG2, IgG3, or IgG4 and/or a constant light chain kappa or lambda region. Such constant domains and regions are well known in the state of the art and e.g. described by Kabat, et al., Sequences of Proteins of Immunological Interest, 5th ed., Public Health Service, National Institutes of Health, Bethesda, Md. (1991).

The term “tertiary structure” as used herein refers to the geometric shape of the antibody according to the invention. The tertiary structure comprises a polypeptide chain backbone comprising the antibody domains, while amino acid side chains interact and bond in a number of ways.

The multivalent antibody according to the invention is produced by recombinant means. Methods for recombinant production of antibodies are widely known in the state of the art and comprise protein expression in prokaryotic and eukaryotic cells with subsequent isolation of the antibody and usually purification to a pharmaceutically acceptable purity. For the expression of antibodies in a host cell, nucleic acids encoding the respective light and heavy chains as described herein are inserted into expression vectors by standard methods. Expression is performed in appropriate prokaryotic or eukaryotic host cells, like CHO cells, NS0 cells, SP2/0 cells, HEK293 cells, COS cells, PER.C6 cells, yeast, or E. coli cells, and the antibody is recovered from the cells (supernatant or cells after lysis). General methods for recombinant production of antibodies are well-known in the state of the art and described, for example, in the review articles of Makrides, S. C., Protein Expr. Purif. 17 (1999) 183-202; Geisse, S., et al., Protein Expr. Purif. 8 (1996) 271-282; Kaufman, R. J., Mol. Biotechnol. 16 (2000) 151-161; Werner, R. G., Drug Res. 48 (1998) 870-880.

“Polynucleotide” or “nucleic acid” as used interchangeably herein, refers to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. A sequence of nucleotides may be interrupted by non-nucleotide components. A polynucleotide may comprise modification(s) made after synthesis, such as conjugation to a label. Other types of modifications include, for example, “caps,” substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotides(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl-, 2′-fluoro- or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs, and basic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), (O)NR2 (“amidate”), P(O)R, P(O)OR′, CO, or CH2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. The term includes vectors that function primarily for insertion of DNA or RNA into a cell (e.g., chromosomal integration), replication of vectors that function primarily for the replication of DNA or RNA, and expression vectors that function for transcription and/or translation of the DNA or RNA. Also included are vectors that provide more than one of the functions as described.

An “expression vector” is a vector are capable of directing the expression of nucleic acids to which they are operatively linked. When the expression vector is introduced into an appropriate host cell, it can be transcribed and translated into a polypeptide. When transforming host cells in methods according to the invention, “expression vectors” are used; thereby the term “vector” in connection with transformation of host cells as described herein means “expression vector”. An “expression system” usually refers to a suitable host cell comprised of an expression vector that can function to yield a desired expression product.

As used herein, “expression” refers to the process by which a nucleic acid is transcribed into mRNA and/or to the process by which the transcribed mRNA (also referred to as transcript) is subsequently being translated into peptides, polypeptides, or proteins. The transcripts and the encoded polypeptides are collectively referred to as gene product. If the polynucleotide is derived from genomic DNA, expression in a eukaryotic cell may include splicing of the mRNA.

The term “transformation” as used herein refers to process of transfer of a vectors/nucleic acid into a host cell. If cells without formidable cell wall barriers are used as host cells, transfection is carried out e.g. by the calcium phosphate precipitation method as described by Graham and Van der Eh, Virology 52 (1978) 546ff. However, other methods for introducing DNA into cells such as by nuclear injection or by protoplast fusion may also be used. If prokaryotic cells or cells which contain substantial cell wall constructions are used, e.g. one method of transfection is calcium treatment using calcium chloride as described by Cohen, F. N, et al., PNAS 69 (1972) 7110 et seq.

The term “host cell” as used in the current application denotes any kind of cellular system which can be engineered to generate the antibodies according to the current invention.

As used herein, the expressions “cell,” “cell line,” and “cell culture” are used interchangeably and all such designations include progeny. Thus, the words “transformants” and “transformed cells” include the primary subject cell and cultures derived therefrom without regard for the number of transfers. It is also understood that all progeny may not be precisely identical in DNA content, due to deliberate or inadvertent mutations. Variant progeny that have the same function or biological activity as screened for in the originally transformed cell are included. Where distinct designations are intended, it will be clear from the context.

Expression in NS0 cells is described by, e.g., Barnes, L. M., et al., Cytotechnology 32 (2000) 109-123; Barnes, L. M., et al., Biotech. Bioeng. 73 (2001) 261-270. Transient expression is described by, e.g., Durocher, Y., et al., Nucl. Acids. Res. 30 (2002) E9. Cloning of variable domains is described by Orlandi, R., et al., Proc. Natl. Acad. Sci. USA 86 (1989) 3833-3837; Carter, P., et al., Proc. Natl. Acad. Sci. USA 89 (1992) 4285-4289; and Norderhaug, L., et al., J. Immunol. Methods 204 (1997) 77-87. A preferred transient expression system (HEK 293) is described by Schlaeger, E.-J., and Christensen, K., in Cytotechnology 30 (1999) 71-83 and by Schlaeger, E.-J., J. Immunol. Methods 194 (1996) 191-199.

Antibodies produced by host cells may undergo post-translational cleavage of one or more, particularly one or two, amino acids from the C-terminus of the heavy chain. Therefore, an antibody produced by a host cell by expression of a specific nucleic acid molecule encoding a full-length heavy chain may include the full-length heavy chain, or it may include a cleaved variant of the full-length heavy chain (also referred to herein as a cleaved variant heavy chain). This may be the case where the final two C-terminal amino acids of the heavy chain are glycine (G446) and lysine (K447, numbering according to Kabat EU index).

Therefore, amino acid sequences of heavy chains including CH3 domains are denoted herein without C-terminal glycine-lysine dipeptide if not indicated otherwise.

Compositions of the invention, such as the pharmaceutical compositions described herein, comprise a population of antibodies of the invention. The population of antibodies may comprise antibodies having a full-length heavy chain and antibodies having a cleaved variant heavy chain. In one embodiment, the population of antibodies consists of a mixture of antibodies having a full-length heavy chain and antibodies having a cleaved variant heavy chain, wherein at least 50%, at least 60%, at least 70%, at least 80% or at least 90% of the antibodies have a cleaved variant heavy chain.

Purification of antibodies (recovering the antibodies from the host cell culture) is performed in order to eliminate cellular components or other contaminants, e.g. other cellular nucleic acids or proteins, by standard techniques, including alkaline/SDS treatment, CsCl banding, column chromatography, agarose gel electrophoresis, and others well known in the art. See Ausubel, F., et al., ed. Current Protocols in Molecular Biology, Greene Publishing and Wiley Interscience, New York (1987). Different methods are well established and widespread used for protein purification, such as affinity chromatography with microbial proteins (e.g. protein A or protein G affinity chromatography), ion exchange chromatography (e.g. cation exchange (carboxymethyl resins), anion exchange (amino ethyl resins) and mixed-mode exchange), thiophilic adsorption (e.g. with beta-mercaptoethanol and other SH ligands), hydrophobic interaction or aromatic adsorption chromatography (e.g. with phenyl-sepharose, aza-arenophilic resins, or m-aminophenylboronic acid), metal chelate affinity chromatography (e.g. with Ni(II)- and Cu(II)-affinity material), size exclusion chromatography, and electrophoretical methods (such as gel electrophoresis, capillary electrophoresis) (Vijayalakshmi, M. A., Appl. Biochem. Biotech. 75 (1998) 93-102).

An “immunoconjugate” is an antibody conjugated to one or more heterologous molecule(s), including but not limited to a detectable label.

Immunoglobulins are glycoproteins specifically binding to target antigens. Bivalent and monospecific immunoglobulins such as naturally occurring forms of IgG have a four-chain structure as their basic unit. They are composed of two identical light chains (L) and two identical heavy chains (H) held together by inter-chain disulfide bonds and by non-covalent interactions. IgM class immunoglobulin represent an exception with respect to the numbers of heavy and light chains, and are not taken into consideration in the following. A light chain is formed by two domains, a variable and a constant one, while one variable domain and three constant domains form the heavy chain. Immunoglobulin sequences are usually numbered according to a common scheme (Kabat-Chothia) aimed at assigning the same number to topologically equivalent residues (Al-Lazikani B et al. J. Mol. Biol. 273 (1997) 927-948). This is a widely adopted standard for ordering and numbering the residues of antibodies in a consistent manner.

In the description related to the modular design of the recombinant antibody as reported herein reference can be made to one or more immunoglobulin domain(s) or region(s) as element(s). In this context, a single element is selected from the group consisting of “CH1”, “CH2”, “CH3”, “CL”, “VH”, “VL”, “<hinge>” and“L”. Each of these single elements may also be referred to as a “core element” of a heavy or light chain in the structural description of a recombinant antibody subject of this disclosure. Several core elements can be combined to a higher-order element, such as (but not limited to) “Fc_{H}”, “Fab_{H}” and “Fab_{L}”, resulting from a combination of core elements as presented in Formula II to X, respectively.

_{N-terminus}[VH-CH1]_{H C-terminus} (a higher-order element Fab_{H}; Formula II),

_{N-terminus}[VH-CL]_{H C-terminus} (a higher-order element Fab_{H}; Formula III),

_{N-terminus}[VL-CL]_{H C-terminus} (a higher-order element Fab_{H}; Formula IV),

_{N-terminus}[VL-CH1]_{H C-terminus} (a higher-order element Fab_{H}; Formula V),

_{N-terminus}[VH-CH1]_{L C-terminus} (a higher-order element Fab_{L}; Formula VI),

_{N-terminus}[VH-CL]_{L C-terminus} (a higher-order element Fab_{L}; Formula VII),

_{N-terminus}[VL-CL]_{L C-terminus} (a higher-order element Fab_{L}; Formula VIII),

_{N-terminus}[VL-CH1]_{L C-terminus} (a higher-order element Fab_{L}; Formula IX),

and

_{N-terminus}<hinge>-CH2-CH3_{C-terminus} (a higher-order element Fc_{H}; Formula X).

The skilled person appreciates that recombinant techniques allow for the construction of different non-naturally-occurring Fab_{H} and Fab_{L} elements as shown above, e.g. but not limited to VL-CL_{H} or VH-CH1_{L}. An antibody with a CL-CH1 replacement in a binding arm exemplifies a so-called CrossMab. CrossMabs are described in detail in WO 2009/080253 and Schaefer, W. et al., PNAS, 108 (2011) 11187-11191.

Generally, unless explicitly indicated otherwise, the orientation of each grouping of elements always is from the N-terminus on the left-hand side to the C-terminus on the right-hand side. In all representations of polypeptide chains with core elements and/or higher-order elements, “-” is a covalent bond within a polypeptide chain. It is further understood that in the description a subset of elements of a heavy chain can be given as an isolated item (exemplified by X=[-L-Fab_{H}] or Y=[Fab_{H}-L-], as the case may be), for the purpose of specific explanation of the elements in the subset and/or its features. Unless indicated otherwise, any subset of the heavy chain which is discussed herein is considered to be a covalently bonded integral part of the complete contiguous heavy chain polypeptide. By the same token, a light chain is always referred to as a set of the elements such as Formula VI to IX, that is to say without additional elements being present in the respective polypeptide chain, if not indicated otherwise.

The skilled person who is familiar with the construction of recombinant immunoglobulins appreciates that each of the core elements “CH1”, “CH2”, “CH3”, “CL”, “VH”, “VL”, “<hinge>” and “L” reflects a functional entity, the functionality of which is not changed by minor alterations of the respective element's amino acid sequence. For example, there can be one or more neutral amino acid exchanges or minor additions or minor deletions of amino acids, specifically terminal additions of 1 to 20 amino acids to any one of the core elements, however provided that despite the presence of said variations a functional immunoglobulin according to the first aspect as provided herein is formed, i.e. alignment of heavy and light chains is not negatively affected and the functionality of the antigen binding sites is not impaired. That is to say, in the presence of neutral amino acid exchanges or minor additions or minor deletions, the alignment and covalent connection of the two heavy chains remain undisturbed, and the alignment and covalent connection of Fab_{H}:Fab_{L} remain undisturbed, and the specificity and sensitivity of antigen binding are unchanged. In this regard, the terms “undisturbed” and “unchanged” have the meaning of being within a range of 95% to 100% of each single respective property compared to the corresponding immunoglobulin without any of the said variations.

In the context of the present disclosure, the basic architecture of a “conventional Ig isotype” is represented by the architecture of a naturally occurring monospecific and bivalent antibody of the isotype selected from the group consisting of IgG, IgA and IgD, wherein the antibody comprises two polypeptides of an immunoglobulin heavy chain of Formula XI

_{N-terminus} Fab_{H}-Fc_{H C-terminus} (Formula XI)

and 2 immunoglobulin light chains Fab_{L}, wherein “-” is a covalent bond within a polypeptide chain; wherein Fc_{H} is an immunoglobulin heavy chain portion comprising a N-terminal hinge domain (=<hinge>), followed by a heavy chain constant domain 2 (=CH2) and a C-terminal heavy chain constant domain 3 (=CH3); wherein each Fab_{H} is a VH-CH1 immunoglobulin heavy chain portion comprising a N-terminal heavy chain variable domain (=VH) and a C-terminal heavy chain constant domain 1 (=CH1); wherein each Fab is a VL-CL immunoglobulin light chain comprising a N-terminal light chain variable domain (=VL) and a C-terminal light chain constant domain (=CL); wherein in the antibody the respective hinge domains, CH2 and CH3 of the two heavy chains are aligned with each other, and the respective hinge domains of the two heavy chains are covalently linked with each other via one or more disulfide bonds; wherein each antigen binding site Fab_{H}:Fab_{L} of the antibody is an aligned pair of a VH-CH1 immunoglobulin heavy chain portion and a VL-CL immunoglobulin light chain, wherein in each pair the respective CL and CH1, and VL and VH are aligned with each other, and in each pair the respective VL-CL and VH-CH1 are covalently linked via a disulfide bond.

Recombinantly engineered examples and established variants of the monospecific and bivalent antibody of conventional Ig isotype known to the skilled person include those with a Fab_{H} that is selected from the group consisting of

_{N-terminus}[VH-CH1]_{H C-terminus} (Formula II),

_{N-terminus}[VH-CL]_{H C-terminus} (Formula III),

_{N-terminus}[VL-CL]_{H C-terminus} (Formula IV), and

_{N-terminus}[VL-CH1]_{H C-terminus} (Formula V),

wherein VH is an immunoglobulin heavy chain variable domain, VL is a immunoglobulin light chain variable domain, CH1 is a immunoglobulin heavy chain constant domain 1, and CL is a immunoglobulin light chain constant domain;

and each Fab_{L} is independently selected from the group consisting of

_{N-terminus}[VH-CH1]_{L C-terminus} (Formula VI),

_{N-terminus}[VH-CL]_{L C-terminus} (Formula VII),

_{N-terminus}[VL-CL]_{L C-terminus} (Formula VIII), and

_{N-terminus}[VL-CH1]_{L C-terminus} (Formula IX);

wherein an antigen binding site Fab_{H}:Fab_{L} of the antibody is an aligned pair, the alignment being signified by “:”, wherein the aligned pair is independently selected from the group consisting of

[VL-CH1]_{H}:[VH-CL]_{L},

[VL-CL]_{H}:[VH-CH1]_{L},

[VH-CH1]_{H}:[VL-CL]_{L}, and

[VH-CL]_{H}:[VL-CH1]_{L},

and wherein in the aligned pair the respective CL and CH1 are covalently linked via a disulfide bond.

Particular technical advantage concerning use of an analyte-specific antibody for detecting the analyte as a target in a complex mixture was found upon experimentally extending the conventional basic architecture by adding/appending further analyte-specific Fab_{H}:Fab_{L} units. Surprisingly, it was found that extension of an antibody by appending further Fab_{H}:Fab_{L} antigen binding sites at the Fc portion provided added benefit with respect to the binding properties of the recombinant antibody. In addition, extending each arm of the antibody by appending further Fab_{H}:Fab_{L} antigen binding sites also improved the binding properties. As one result, compared to a conventional IgG molecule, a single multivalent recombinant antibody is characterized by a higher value concerning the ratio of the surface of antigen-binding regions versus the surface of non-antigen-binding regions. Particular advantage has been observed in an improved signal-to-noise ratio when an antibody as disclosed herein is used as capture and/or detection agent, e.g. in a sandwich immunoassay for detecting an antigen.

Therefore, as a first aspect related to all other aspects and embodiments reported herein, the present disclosure provides a chimeric or non-chimeric multivalent recombinant antibody, wherein the antibody comprises p light chain polypeptides Fab_{L} and a dimer of two heavy chain polypeptides, wherein each heavy chain polypeptide has a structure of Formula I

_{N-terminus}[Fab_{H}-L-]ₙFab_{H}-L-dd(Fc_{H})[-L-Fab_{H}]_{m C-terminus} (Formula I)

wherein
(i) p is a value selected from the group consisting of 6, 8, and 10,
each of m and n is selected independently from an integer of 1 to 3, and
each of m and n is selected such that the value of p equals (2+2*(n+m));
(j) “-” is a covalent bond within a polypeptide chain;
(k) each L is optional and, if present, is an independently selected variable linker amino acid sequence;
(l) each dd(Fc_{H}) is a heavy chain dimerization region [of a heavy chain of a non-antigen binding immunoglobulin region;
(m) in the dimer the two dd(Fc_{H}) are aligned with each other in physical proximity;
(n) each Fab_{H} is independently selected from A_{H} and B_{H}, wherein A_{H} and B_{H} are different, and A_{H} and B_{H} are independently selected from the group consisting of

_{N-terminus}[VH-CH1]_{H C-terminus} (Formula II),

_{N-terminus}[VH-CL]_{H C-terminus} (Formula III),

_{N-terminus}[VL-CL]_{H C-terminus} (Formula IV), and

_{N-terminus}[VL-CH1]_{H C-terminus} (Formula V),
wherein
VH is a N-terminal immunoglobulin heavy chain variable domain,
VL is a N-terminal immunoglobulin light chain variable domain,
CH1 is a C-terminal immunoglobulin heavy chain constant domain 1, and
CL is a C-terminal immunoglobulin light chain constant domain;
(o) each Fab_{L} is independently selected from A_{L} and B_{L}, wherein A_{L} and B_{L} are different, and A_{L} and B_{L} are independently selected from the group consisting of

_{N-terminus}[VH-CH1]_{L C-terminus} (Formula VI),

_{N-terminus}[VH-CL]_{L C-terminus} (Formula VII),

_{N-terminus}[VL-CL]_{L C-terminus} (Formula VIII), and

_{N-terminus}[VL-CH1]_{L C-terminus} (Formula IX);
(p) each antigen binding site Fab_{H}:Fab_{L} of the antibody is an aligned pair (the alignment being signified by “:”), wherein each aligned pair is independently selected from the group consisting of A_{H}:A_{L} and B_{H}:B_{L}, wherein A_{H}:A_{L} and B_{H}:B_{L} are selected independently from the group consisting of

[VL-CH1]_{H}:[VH-CL]_{L},

[VL-CL]_{H}:[VH-CH1]_{L},

[VH-CH1]_{H}:[VL-CL]_{L}, and

[VH-CL]_{H}:[VL-CH1]_{L},

and wherein in each aligned pair the respective CL and CH1 are covalently linked via a disulfide bond.

The recombinant antibody as disclosed herein is not bivalent as a conventional antibody of one of the immunoglobulin classes IgA, IgD, IgE or IgG, but it is multivalent. In a recombinant multivalent antibody as disclosed herewith, the light chains are similar or identical to the light chains of naturally occurring immunoglobulins. Importantly, it is the design of the recombinant immunoglobulin heavy chain that realizes multivalent antigen binding, by providing a plurality of Fab_{H} elements in each heavy chain. The novel recombinant antibody as reported in all aspects and embodiments herein is characterized by a modular design which combines 4 or more antigen binding sites (=Fab=Fab_{H}:Fab_{L}) in a single antibody molecule. More specifically, presented herein is a recombinant multivalent antibody having a number of antigen binding sites represented by the value of p presented above in Formula I, wherein p is an integer which can be selected from the group consisting of 6, 8, and 10, and optionally 12. That is to say, in a single heavy chain the number of comprised Fab_{H} elements is p/2, i.e. an integer selected from the group consisting of 3, 4, 5, and optionally 6.

The values of m and n are selected independently, and each of m and n is selected such that the value of p equals (2+2*(n+m)). In a embodiment, the value of m is selected from the group of integers consisting of 0, 1, 2, 3, and 4. More specifically, m is selected from the group of integers consisting of 1, 2, 3, and 4. Even more specifically, m is selected from the group of integers consisting of 1, 2, and 3. In another embodiment, the value of n is selected from the group of integers consisting of 0, 1, 2, 3, and 4. More specifically, n is selected from the group of integers consisting of 1, 2, 3, and 4. Even more specifically, n is selected from the group of integers consisting of 1, 2, and 3.

By way of example, in an embodiment wherein p is 6, both m and n are 1. In an embodiment wherein p is 8, either n is 1 and m is 2 or n is 2 and m is 2. In an embodiment wherein p is 10, n is 1, 2, or 3, and m is 3, 2 or 1, respectively. In an embodiment wherein p is 12, n is 1, 2, 3, or 4 and m is 4, 3, 2 or 1, respectively.

The recombinant antibody as related to all aspects and embodiments disclosed herein comprises—and in an embodiment exclusively consists of—immunoglobulin domains, immunoglobulin elements and immunoglobulin regions, according to the modular design as disclosed in here.

The recombinant antibody as related to all aspects and embodiments disclosed herein comprises two aligned heavy chains of Formula I. Each heavy chain is made up of structural elements, presented in the order starting with the N-terminus, and listing the elements from there towards and to the C-terminus of the heavy chain.

In an embodiment, a recombinant antibody as related to all aspects and embodiments disclosed herein is chimeric and comprises elements from different species of origin. In another embodiment, a recombinant antibody as related to all aspects and embodiments disclosed herein is a non-chimeric antibody which contains elements that are derived from the same species.

The recombinant antibody as provided in all aspects and embodiments herein is characterized by a modular design which combines structural elements of Fc_{H} constant domains of immunoglobulin heavy chains that are known to the art. In an immunoglobulin molecule in its native (i.e. non engineered) form, a heavy chain Fc_{H} polypeptide is made up of constant domains CH2 and CH3 as core elements. N-terminally appended to the CH2 domain is a <hinge> domain providing cysteine —SH groups for heavy chain crosslinks. In a specific embodiment of the recombinant antibody, the Fc_{H} portion comprises the arrangement of domains according to Formula X, <hinge>-CH2-CH3 of an immunoglobulin of a class selected from the group consisting of IgG, IgA and IgD. In a more specific embodiment, the Fc_{H} portion is represented by an amino acid sequence having its origin in a mammalian species selected from the group consisting of human, mouse, rat, sheep, and rabbit.

In a more general way, a Fc_{H} higher-order element is an embodiment of a heavy chain dimerization domain of a heavy chain of a non-antigen binding immunoglobulin region, referred to herein as dd(Fc_{H}). Of central importance is that dd(Fc_{H}) facilitates alignment and connection of the two heavy chain polypeptides which are part of the multivalent recombinant antibody as disclosed herein. Connection in this respect can be by physical forces entirely, e.g. in an embodiment the dd(Fc_{H}) comprises a single CH3 element capable of forming a CH3/CH3 complex as in the paired heavy chains of an IgG molecule. An embodiment of dd(Fc_{H}) is a domain comprising one or more elements selected from the group consisting of CH3, <hinge>-CH3, and <hinge>-CH2-CH3. Another embodiment of dd(Fc_{H}) is a domain consisting of one element selected from the group consisting of CH3, <hinge>-CH3, and <hinge>-CH2-CH3. Provided that a hinge region is present, the connection of the two heavy chains is not only by physical forces but also by disulfide bridges between cysteine residues of the hinge region in the first and the second heavy chain of the multivalent recombinant antibody as disclosed herein.

The Fc_{H} portion of the heavy chain can be extended at the N-terminus by a linker amino acid sequence L which is optional and, if present, is an independently selected variable linker amino acid sequence.

Generally, in the context of the present disclosure and related to all aspects and embodiments herein, a “linker amino acid sequence” denoted by “L” is a peptide sequence comprising 1 to 60 amino acid residues that connects two domains of a polypeptide that comprises a plurality of domains, specifically a plurality of different domains. In the heavy chain of the recombinant antibody disclosed herein, linker amino acid sequences are contemplated as optional elements at different locations as presented in Formula I.

A linker amino acid sequence is typically composed of flexible residues like glycine and serine so that the adjacent domains of the polypeptide are free to move relative to one another. Longer sequences can be particularly useful when it is necessary to ensure that two adjacent domains do not sterically interfere with one another. In a specific embodiment the linker amino acid sequence comprises, more specifically consists of, glycine and serine residues. The amino acids glycine and serine are zwitterionic and hydrophilic. These properties make them a frequent choice for a repetitive linker sequence. Thus, in a yet more specific embodiment, each linker amino acid sequence in the heavy chain of the recombinant antibody related to all aspects and embodiments as disclosed herein comprises an independently selected variable linker amino acid sequence selected from the group consisting of Formula XII,

_{N-terminus}(GᵤS_{q})_{r C-terminus} (Formula XII)

wherein u is an integer selected from 1 to 10, q is an integer from 1 to 5, and r is an integer from 1 to 10. In yet an even more specific embodiment, each linker amino acid sequence comprises an independently selected variable linker amino acid sequence of Formula XII, wherein u is 3 or 4, q is 1 and r is selected from the group consisting of 3, 4, 5, and 6. A very specific linker amino acid sequence comprises, even more specifically consists of, the amino acid sequence GGGSGGGSGGGSGGGS (SEQ ID NO:1). The skilled person in this context appreciates alternative glycine and serine containing repetitive sequences, too, which suitably serve the same technical purpose. In yet another specific embodiment a selected linker amino acid sequence is (GSAT)₁, (GSAT)₂, (GSAT)₃ or (GSAT)₄. In yet another specific embodiment a selected linker amino acid sequence is (SEG)₁, (SEG)₂, (SEG)₃ or (SEG)₄.

In a different specific embodiment a selected linker amino acid sequence is (EAAAR)₁, (EAAAR)₂, (EAAAR)₃, (EAAAR)₄ or (EAAAR)₅ (Merutka G, et al. Biochem. 30 (1991) 4245-4248; Sommese R F, et al. Protein Sci. 19 (2010) 2001-2005; Yan W et al. Biochem. 46 (2007) 8517-8524). The skilled person in this context appreciates that the EAAR elements are examples of a more rigid linker to keep the two domains attached at either end from coming closer together.

Extending the Fc_{H} portion N-terminally a CH1 domain connected to the hinge domain (<hinge>), optionally via a linker L. However, in one embodiment a heavy chain of Formula I contains a contiguous CH1-<hinge>-CH2-CH3 portion derived from an immunoglobulin of an isotype selected from the group consisting of IgG, IgA and IgD. In a more specific embodiment, the CH1 domain and the <hinge>-CH2-CH3 portion are represented by an amino acid sequence having its origin in a mammalian species selected from the group consisting of human, mouse, rat, sheep, and rabbit. Exemplary CH1 and <hinge>-CH2-CH3 portions include the respective amino acid sequences depicted in Table A.

TABLE A

| Species | Amino acid | Amino acid sequence |
|---|---|---|
| origin and | sequence for | for <hinge>-CH2-CH3 |
| Ig isotype | CH1 domain | portion (Fc_{H}) |
| Murine IgG1 | AKTTPPSVYPLAPGS | CKPCICTVPEVSSVFIFPPK |
| | AAQTNSMVTLGCLVK | PKDVLTITLTPKVTCVVVDI |
| | GYFPEPVTVTWNSGS | SKDDPEVQFSWFVDDVEVHT |
| | LSSGVHTFPAVLQSD | AQTQPREEQFNSTFRSVSEL |
| | LYTLSSSVTVPSSTW | PIMHQDWLNGKEFKCRVNSA |
| | PSETVTCNVAHPASS | AFPAPIEKTISKTKGRPKAP |
| | TKVDKKIVPRDCG | QVYTIPPPKEQMAKDKVSLT |
| | (SEQ ID NO: 2) | CMITDFFPEDITVEWQWNGQ |
| | | PAENYKNTQPIMDTDGSYFV |
| | | YSKLNVQKSNWEAGNTFTCS |
| | | VLHEGLHNHHTEKSLSHSPG |
| | | K (SEQ ID NO: 3) |
| Murine IgG2 | AKTTAPSVYPLAPVC | PCKCPAPNLLGGPSVFIFPP |
| | GDTTGSSVTLGCLVK | KIKDVLMISLSPIVTCVVVD |
| | GYFPEPVTLTWNSGS | VSEDDPDVQISWFVNNVEVH |
| | LSSGVHTFPAVLQSD | TAQTQTHREDYNSTLRVVSA |
| | LYTLSSSVTVTSSTW | LPIQHQDWMSGKEFKCKVNN |
| | PSQSITCNVAHPASS | KDLPAPIERTISKPKGSVRA |
| | TKVDKKIEPRGPTIK | PQVYVLPPPEEEMTKKQVTL |
| | PCP | TCMVTDFMPEDIYVEWTNNG |
| | (SEQ ID NO: 4) | KTELNYKNTEPVLDSDGSYF |
| | | MYSKLRVEKKNWVERNSYSC |
| | | SVVHEGLHNHHTTKSFSRTP |
| | | GK (SEQ ID NO: 5) |
| Murine IgG3 | TTTAPSVYPLVPGCS | EPRIPKPSTPPGSSCPAGNI |
| | DTSGSSVTLGCLVKG | LGGPSVFIFPPKPKDALMIS |
| | YFPEPVTVKWNYGAL | LTPKVTCVVVDVSEDDPDVH |
| | SSGVRTVSSVLQSGF | VSWFVDNKEVHTAWTQPREA |
| | YSLSSLVTVPSSTWP | QYNSTFRVVSALPIQHQDWM |
| | SQTVICNVAHPASKT | RGKEFKCKVNNKALPAPIER |
| | ELIKRI | TISKPKGRAQTPQVYTIPPP |
| | (SEQ ID NO: 6) | REQMSKKKVSLTCLVTNFFS |
| | | EAISVEWERNGELEQDYKNT |
| | | PPILDSDGTYFLYSKLTVDT |
| | | DSWLQGEIFTCSVVHEALHN |
| | | HHTQKNLSRSPGK |
| | | (SEQ ID NO: 7) |
| Human IgG1 | ASTKGPSVFPLAPSS | KTHTCPPCPAPELLGGPSVF |
| | KSTSGGTAALGCLVK | LFPPKPKDTLMISRTPEVTC |
| | DYFPEPVTVSWNSGA | VVVDVSHEDPEVKFNWYVDG |
| | LTSGVHTFPAVLQSS | VEVHNAKTKPREEQYNSTYR |
| | GLYSLSSVVTVPSSS | VVSVLTVLHQDWLNGKEYKC |
| | LGTQTYICNVNHKPS | KVSNKALPAPIEKTISKAKG |
| | NTKVDKKVEPKSCD | QPREPQVYTLPPSRDELTKN |
| | (SEQ ID NO: 8) | QVSLTCLVKGFYPSDIAVEW |
| | | ESNGQPENNYKTTPPVLDSD |
| | | GSFFLYSKLTVDKSRWQQGN |
| | | VFSCSVMHEALHNHYTQKSL |
| | | SLSPGK |
| | | (SEQ ID NO: 9) |
| Human IgG2 | ASTKGPSVFPLAPCS | CVECPPCPAPPVAGPSVFLF |
| | RSTSESTAALGCLVK | PPKPKDTLMISRTPEVTCVV |
| | DYFPEPVTVSWNSGA | VDVSHEDPEVQFNWYVDGVE |
| | LTSGVHTFPAVLQSS | VHNAKTKPREEQFNSTFRVV |
| | GLYSLSSVVTVPSSN | SVLTVVHQDWLNGKEYKCKV |
| | FGTQTYTCNVDHKPS | SNKGLPAPIEKTISKTKGQP |
| | NTKVDKTVERKC | REPQVYTLPPSREEMTKNQV |
| | (SEQ ID NO: 10) | SLTCLVKGFYPSDIAVEWES |
| | | NGQPENNYKTTPPMLDSDGS |
| | | FFLYSKLTVDKSRWQQGNVF |
| | | SCSVMHEALHNHYTQKSLSL |
| | | SPGK (SEQ ID NO: 11) |
| Human IgG3 | ASTKGPSVFPLAPCS | RCPEPKSCDTPPPCPRCPEP |
| | RSTSGGTAALGCLVK | KSCDTPPPCPRCPEPKSCDT |
| | DYFPEPVTVSWNSGA | PPPCPRCPAPELLGGPSVFL |
| | LTSGVHTFPAVLQSS | FPPKPKDTLMISRTPEVTCV |
| | GLYSLSSVVTVPSSS | VVDVSHEDPEVQFKWYVDGV |
| | LGTQTYTCNVNHKPS | EVHNAKTKPREEQYNSTFRV |
| | NTKVDKRVELKTPLG | VSVLTVLHQDWLNGKEYKCK |
| | DTTHTCP | VSNKALPAPIEKTISKTKGQ |
| | (SEQ ID NO: 12) | PREPQVYTLPPSREEMTKNQ |
| | | VSLTCLVKGFYPSDIAVEWE |
| | | SSGQPENNYNTTPPMLDSDG |
| | | SFFLYSKLTVDKSRWQQGNI |
| | | FSCSVMHEALHNRFTQKSLS |
| | | LSPGK |
| | | (SEQ ID NO: 13) |
| Human IgG4 | ASTKGPSVFPLAPCS | PHAHHAQAPEFLGGPSVFLF |
| | RSTSESTAALGCLVK | PPKPKDTLMISRTPEVTCVV |
| | DYFPEPVTVSWNSGA | VDVSQEDPEVQFNWYVDGVE |
| | LTSGVHTFPAVLQSS | VHNAKTKPREEQFNSTYRVV |
| | GLYSLSSVVTVPSSS | SVLTVLHQDWLNGKEYKCKV |
| | LGTKTYTCNVDHKPS | SNKGLPSSIEKTISKAKGQP |
| | NTKVDKRVSPNMV | REPQVYTLPPSQEEMTKNQV |
| | (SEQ ID NO: 14) | SLTCLVKGFYPSDIAVEWES |
| | | NGQPENNYKTTPPVLDSDGS |
| | | FFLYSRLTVDKSRWQEGNVF |
| | | SCSVMHEALHNHYTQKSLSL |
| | | SLGK (SEQ ID NO: 15) |
| Rabbit IgG | GQPKAPSVFPLAPCC | KPTCPPPELLGGPSVFIFPP |
| | GDTPSSTVTLGCLVK | KPKDTLMISRTPEVTCVVVD |
| | GYLPEPVTVTWNSGT | VSQDDPEVQFTWYINNEQVR |
| | LTNGVRTFPSVRQSS | TARPPLREQQFNSTIRVVST |
| | GLYSLSSVVSVTSSS | LPIAHQDWLRGKEFKCKVHN |
| | QPVTCNVAHPATNTK | KALPAPIEKTISKARGQPLE |
| | VDKTVAPSTCS | PKVYTMGPPREELSSRSVSL |
| | (SEQ ID NO: 16) | TCMINGFYPSDISVEWEKNG |
| | | KAEDNYKTTPAVLDSDGSYF |
| | | LYNKLSVPTSEWQRGDVFTC |
| | | SVMHEALHNHYTQKSISRSP |
| | | GK (SEQ ID NO: 17) |

Despite amino acid differences between the isotypes and the subclasses within an isotype, each CH region within an immunoglobulin heavy chain folds into a constant structure consisting of a three strand-four strand beta sheet linked together by an intra-chain disulfide bond (Schroeder H. W. & Cavacini L. J Allergy Clin Immunol. (2010) 125: S41-S52).

The modular architecture presented here also contemplates that within the heavy chain of the recombinant antibody as provided in all aspects and embodiments herein, one or more CH1 element(s) may be substituted by a CL element. Exemplary CL elements include the respective amino acid sequences depicted in Table B. Immunoglobulin light chains are classified as kappa or lambda according to their serological and sequence properties. While the table displays amino acids for CL kappa domains, it is understood that CL lambda domains are not excluded from the choices of constant elements to build a heavy chain portion of a Fab_{H}.

TABLE B

| Species origin | Amino acid sequence for |
|---|---|
| and Ig isotype | CL kappa domain |
| Murine IgG | APTVSIFPPSSEQLTSGGASVVCFLNNFYP |
| | KDINVKWKIDGSERQNGVLNSWTDQDSKDS |
| | TYSMSSTLTLTKDEYERHNSYTCEATHKTS |
| | TSPIVKSFNRNEC (SEQ ID NO: 18) |
| Human IgG | APTVSIFPPSSEQLTSGGASVVCFLNNFYP |
| | KDINVKWKIDGSERQNGVLNSWTDQDSKDS |
| | TYSMSSTLTLTKDEYERHNSYTCEATHKTS |
| | TSPIVKSFNRNEC (SEQ ID NO: 19) |
| Rabbit IgG | APTVLIFPPAADQVATGTVTIVCVANKYFP |
| | DVTVTWEVDGTTQTTGIENSKTPQNSADCT |
| | YNLSSTLTLTSTQYNSHKEYTCKVTQGTTS |
| | VVQSFNRGDC (SEQ ID NO: 20) |

The skilled person is aware of the fact that minor alterations of the amino acid sequences representing a CH region are possible and will not interfere with the structural features of these domains.

The variable domains determine antigen specificity. Most of the diversity of the variable domains resides in three regions from each (heavy and light) chain, called the hypervariable regions or CDRs. These are named according to the chain they belong to and the order they appear in the sequence (L1, L2, L3, H1, H2 and H3). The regions between the CDRs in the variable region are called the framework regions (FW).

C-terminally, the Fc_{H} portion of the recombinant antibody of all aspects and embodiments presented herein can be extended by a variable domain being part of a further Fab_{H} portion. Between the CH3 element of a Fab_{H} portion and a neighboring variable domain a linker amino acid sequence L is optionally located. Specific details and embodiments concerning the linker amino acid sequence have been given above.

Within the heavy chain of the recombinant antibody each CH1 or CL element is combined with a VH or a VL element thereby forming the heavy chain portion of a Fab_{H}. VH and VL elements are selected from pre-existing molecularly characterized monoclonal antibodies which are directed against a desired antigen. In this context, “molecularly characterized” means that the amino acid sequences of the VH and VL domains of a selected pre-existing monoclonal antibody have been determined, as the essential basis for antibody engineering. Thus, in all aspects and embodiments a Fab_{H} element is selected from the group consisting of

_{N-terminus}[VH-CH1]_{H C-terminus} (Formula II),

_{N-terminus}[VH-CL]_{H C-terminus} (Formula III),

_{N-terminus}[VL-CL]_{H C-terminus} (Formula IV), and

_{N-terminus}[VL-CH1]_{H C-terminus} (Formula V),

In a specific embodiment of all aspects of the multivalent recombinant antibody disclosed herein, the two heavy chains are identical or non-identical. An example for two non-identical heavy chains is the knob-in-hole configuration which directs the pairing and alignment of the two heavy chains during the intracellular assembly of the antibody. In another embodiment, one heavy chain has appended to its C- or N-terminus a tag. In a more specific embodiment the tag is an affinity tag such as a Histidine tag known to the art. In another embodiment, the tag is attached C-terminally and comprises positively charged amino acids. In other specific embodiments the two heavy chains are identical.

In a specific embodiment of all aspects of the multivalent recombinant antibody disclosed herein, the multivalent antibody is monospecific and the antigen binding sites are identical or different.

In a specific embodiment of all aspects of the multivalent recombinant antibody disclosed herein, the multivalent antibody is monospecific and all antigen binding sites are derived from one single origin monospecific monoclonal antibody and represent the antigen binding site Fab_{H}:Fab_{L} of the origin monoclonal antibody. Accordingly, the multivalent recombinant antibody comprises either A_{H}:A_{L} or B_{H}:B_{L}.

In another specific embodiment of all aspects of the multivalent recombinant antibody disclosed herein, the multivalent antibody is monospecific and the antigen binding site of A_{H}:A_{L} is capable of binding to a first epitope and the antigen binding site B_{H}:B_{L} is capable of binding to a second epitope, wherein the first and the second epitopes are identical. In this embodiment the structural composition of the two antigen binding sites is different, and they are derived from two different origin monospecific monoclonal antibodies of which each binds to the same epitope, however with differences in their respective binding pockets. In yet another specific embodiment, the antibody is monospecific and the antigen binding sites are identical or different, and the antigen binding sites are capable of specifically binding to an epitope comprised in a single molecule or in different molecules.

In another specific embodiment of all aspects of the multivalent recombinant antibody disclosed herein, the multivalent antibody is bispecific, that is the antibody contains A_{H}:A_{L} and B_{H}:B_{L}, and a first antigen binding site is capable of specifically binding to a first epitope, and a second antigen binding site is capable of specifically binding to a different second epitope, wherein the first epitope and the second epitope are comprised in a single molecule.

In another specific embodiment of all aspects of the multivalent recombinant antibody disclosed herein, the multivalent antibody is bispecific, that is the antibody contains A_{H}:A_{L} and B_{H}:B_{L}, and a first antigen binding site is capable of specifically binding to a first epitope, and a second antigen binding site is capable of specifically binding to a different second epitope, wherein the first epitope is comprised in a first molecule and the second epitope is comprised in a second molecule. In a specific embodiment, the first and the second molecules are identical or non-identical. In another specific embodiment, the two molecules are comprised in an aggregate or in a complex.

In another specific embodiment of all aspects of the multivalent recombinant antibody disclosed herein, the multivalent antibody is coupled to a detectable label. In principle, all labels known to the person skilled in the art of designing immunoassays are possible. Specifically, a detectable label is an enzyme capable of catalyzing the reaction of a substrate, wherein the reacted substrate is a water-soluble or -insoluble dye or colorant. Alternatively, the enzyme catalyzes the reaction of a substrate, wherein the reaction of the substrate generates photon emissions. In a preferred embodiment, the label is a chemiluminescent agent, more specifically an electrochemiluminescent compound capable of being covalently connected to the multivalent recombinant antibody. A specific electrochemiluminescent compound is a Ruthenium-containing (Ruthenium complex) compound as described e.g. in Staffilani M. et al. Inorg. Chem. 42 (2003) 7789-7798, and other Ruthenium- or Iridium containing (Ruthenium or Iridium complex) compounds known to the art.

As a second aspect related to all other aspects and embodiments reported herein, the present disclosure provides the use of a multivalent antibody in an assay for the detection of an antigen, wherein the antibody is a chimeric or non-chimeric multivalent recombinant antibody, wherein the antibody comprises p light chain polypeptides Fab_{L} and a dimer of two heavy chain polypeptides, wherein each heavy chain polypeptide has a structure of Formula I

_{N-terminus}[Fab_{H}-L-]ₙFab_{H}-L-dd(Fc_{H})[-L-Fab_{H}]_{m C-terminus} (Formula I)

wherein
(q) p is a value selected from the group consisting of 6, 8, and 10,
each of m and n is selected independently from an integer of 1 to 3, and
each of m and n is selected such that the value of p equals (2+2*(n+m));
(r) “-” is a covalent bond within a polypeptide chain;
(s) each L is optional and, if present, is an independently selected variable linker amino acid sequence;
(t) each dd(Fc_{H}) is a heavy chain dimerization region of a heavy chain of a non-antigen binding immunoglobulin region;
(u) in the dimer the two dd(Fc_{H}) are aligned with each other in physical proximity;
(v) each Fab_{H} is independently selected from A_{H} and B_{H}, wherein A_{H} and B_{H} are different, and A_{H} and B_{H} are independently selected from the group consisting of

_{N-terminus}[VH-CH1]_{H C-terminus} (Formula II),

_{N-terminus}[VH-CL]_{H C-terminus} (Formula III),

_{N-terminus}[VL-CL]_{H C-terminus} (Formula IV), and

_{N-terminus}[VL-CH1]_{H C-terminus} (Formula V),
wherein
VH is a N-terminal immunoglobulin heavy chain variable domain,
VL is a N-terminal immunoglobulin light chain variable domain,
CH1 is a C-terminal immunoglobulin heavy chain constant domain 1, and
CL is a C-terminal immunoglobulin light chain constant domain;
(w) each Fab_{L} is independently selected from A_{L} and B_{L}, wherein A_{L} and B_{L} are different, and A_{L} and B_{L} are independently selected from the group consisting of

_{N-terminus}[VH-CH1]_{L C-terminus} (Formula VI),

_{N-terminus}[VH-CL]_{L C-terminus} (Formula VII),

_{N-terminus}[VL-CL]_{L C-terminus} (Formula VIII), and

_{N-terminus}[VL-CH1]_{L C-terminus} (Formula IX);
(x) each antigen binding site Fab_{H}:Fab_{L} of the antibody is an aligned pair (the alignment being signified by “:”), wherein each aligned pair is independently selected from the group consisting of A_{H}:A_{L} and B_{H}:B_{L}, wherein A_{H}:A_{L} and B_{H}:B_{L} are selected independently from the group consisting of

[VL-CH1]_{H}:[VH-CL]_{L},

[VL-CL]_{H}:[VH-CH1]_{L},

[VH-CH1]_{H}:[VL-CL]_{L}, and

[VH-CL]_{H}:[VL-CH1]_{L},

and wherein in each aligned pair the respective CL and CH1 are covalently linked via a disulfide bond.

In a specific embodiment of the use as disclosed in here, the assay is a sandwich assay in which the antigen is bound by a first capture antibody and a second detector antibody. In a specific embodiment of the use according to all other aspects and embodiments as disclosed herein, the multivalent antibody is a capture antibody. In another specific embodiment of the use according to all other aspects and embodiments as disclosed herein, the multivalent antibody is a labeled detector antibody.

A third aspect related to all other aspects and embodiments reported herein, the present disclosure provides a kit comprising a chimeric or non-chimeric multivalent recombinant antibody as disclosed in the first aspect of the present disclosure. In a specific embodiment, the kit further comprises a detectable label. In yet another embodiment, the detectable label is attached to the multivalent recombinant antibody. In a further embodiment, the kit additionally comprises magnetic particles coated with a specific binding partner anti-X, and a capturing agent capable of binding to an antigen to which also the multivalent recombinant antibody binds, wherein the capturing agent is conjugated with X, and X and anti-X are capable of forming a stable complex.

A fourth aspect related to all other aspects and embodiments reported herein, the present disclosure provides a method for detecting an antigen, the method comprising the steps of contacting a multivalent recombinant antibody as disclosed in the first aspect of the present disclosure with the antigen, thereby forming a complex of antigen and multivalent recombinant antibody, followed by detecting formed complex, thereby detecting the antigen. In a specific embodiment, the method comprises the steps of (a) mixing a multivalent recombinant antibody according to the present disclosure with a liquid sample suspected of containing the antigen, (b) incubating the sample and the multivalent recombinant antibody of step (a), thereby forming a complex of antigen and multivalent recombinant antibody if antigen is present and accessible for contact with the multivalent recombinant antibody during the incubation, (c) detecting complex formed in step (b), thereby detecting the antigen. Such a detection in another specific embodiment is qualitative, i.e. detects presence or absence of the antigen in the liquid sample. In another embodiment, the detection is quantitative, i.e. detects the amount of the antigen in the liquid sample which, in an even more specific embodiment is suspected of containing the antigen.

In an embodiment the liquid sample is an aqueous sample, more specifically a body fluid, even more specifically a body fluids selected from the group consisting of whole blood, serum, hemolyzed blood, plasma, serum, urine, synovial fluid, liquor cerebro-spinalis, lacrimal fluid, sputum, saliva, breath condensate, bronchio-alveolar lavage, semen, female ejaculate, vaginal lubrication, breast milk, breast aspirate, amniotic fluid, lymph, interstitial fluid, mucus, suspension of feces or cleared supernatant thereof, cell homogenate or cleared supernatant thereof, exudate, sweat, peritoneal fluid, bile, pleural fluid, pericardial fluid, and the like.

In yet another specific embodiment of the fourth aspect as disclosed herein, the method for detecting the antigen comprises the steps of (a) mixing a multivalent recombinant antibody according to the present disclosure with a liquid sample suspected of containing the antigen, (b) incubating the sample and the multivalent recombinant antibody of step (a), thereby forming a complex of antigen and multivalent recombinant antibody if antigen is present and accessible for contact with the multivalent recombinant antibody during the incubation, (c) immobilizing complex formed in step (b), and (d) detecting immobilized complex, thereby detecting the antigen, quantitatively or qualitatively.

In yet another specific embodiment of the fourth aspect as disclosed herein, the method comprises the steps of (a) mixing a labeled multivalent recombinant antibody according to the present disclosure with a liquid sample suspected of containing the antigen, (b) incubating the sample and the labeled multivalent recombinant antibody of step (a), thereby forming a complex of antigen and labeled multivalent recombinant antibody if antigen is present and accessible for contact with the labeled multivalent recombinant antibody during the incubation, (c) immobilizing complex formed in step (b), and (d) detecting immobilized label, thereby detecting the antigen. In a further specific embodiment this method is advantageously performed using a kit of the present disclosure.

In an embodiment, a detectable label such as, but not limited to, a label capable of being detected by way of electrochemiluminescence is attached to the multivalent recombinant antibody, and the method comprises the steps of (a) mixing a labeled multivalent recombinant antibody according to the present disclosure with a liquid sample suspected of containing the antigen, (b) incubating the sample and the labeled multivalent recombinant antibody of step (a), magnetic particles coated with a specific binding partner anti-X, and a capturing agent capable of binding to an antigen to which also the multivalent recombinant antibody binds, wherein the capturing reagent is conjugated with X, thereby forming a sandwich complex of coated magnetic particles, capturing reagent, antigen and labeled multivalent recombinant antibody if antigen is present and accessible for contact with both the labeled multivalent recombinant antibody and the capture reagent during the incubation, (c) immobilizing the sandwich complex formed in step (b), and (d) detecting immobilized label, thereby detecting the antigen. In a further specific embodiment this method is advantageously performed using a kit of the present disclosure.

In yet another embodiment, the method comprises the steps of adding a labeled multivalent recombinant antibody according to the present disclosure to a solid phase suspected of containing the antigen on its surface, incubating the solid phase and the labeled multivalent recombinant antibody of step (a), thereby forming a complex of antigen and the labeled multivalent recombinant antibody if antigen is present and accessible for contact with the labeled multivalent recombinant antibody during the incubation, followed by washing the solid phase, thereby removing not complexed labeled multivalent recombinant antibody, followed by detecting label on the solid phase, thereby detecting the antigen. In a more specific embodiment, the solid phase is capable of capturing the antigen, and prior to step (a) a step of contacting the solid phase with a liquid sample suspected of containing the antigen is performed, wherein antigen is captured by the solid phase if antigen is present and accessible for capture by the solid phase.

The following examples and figures are provided to aid the understanding of the present invention, the true scope of which is set forth in the appended claims. It is understood that modifications can be made in the procedures set forth without departing from the spirit of the invention.

### Example 1

General Knowledge, Methods and Techniques

Standard procedures known to the art were used. Molecular cloning methods are provided in e.g. Sambrook J. “The condensed protocols from Molecular cloning: A laboratory manual” Cold Spring Harbor Laboratory Press (2006). Recombinant antibody production techniques are provided in e.g. Ossipow V. & Fischer N. (eds.) “Monoclonal Antibodies”, Methods in Molecular Biology Vol. 1131 (2014) Springer. Protein chemistry techniques are provided in e.g. Hermanson, G. “Bioconjugate Techniques” 3rd Edition (2013) Academic Press. Bioinformatics methods are provided in e.g. Keith J. M. (ed.) “Bioinformatics” Vol. I and Vol. II, Methods in Molecular Biology Vol. 1525 and Vol. 1526 (2017) Springer, and in Martin, A. C. R. & Allen, J. “Bioinformatics Tools for Analysis of Antibodies” in: Dübel S. & Reichert J. M. (eds.) “Handbook of Therapeutic Antibodies” Wiley-VCH (2014). Immunoassays and related methods are provided in e.g. Wild D. (ed.) “The Immunoassay Handbook” 4th Edition (2013) Elsevier. Ruthenium complexes as electrochemiluminescent labels are provided in e.g. Staffilani M. et al. Inorg. Chem. 42 (2003) 7789-7798. Typically, for the performance of electrochemiluminescence (ECL) based immunoassays an Elecsys 2010 analyzer or a successor system was used, e.g. a Roche analyzer (Roche Diagnostics GmbH, Mannheim Germany) such as E170, cobas e 601 module, cobas e 602 module, cobas e 801 module, and cobas e 411, and Roche Elecsys assays designed for these analyzers, each used under standard conditions, if not indicated otherwise.

### Example 2

Previously Established Workflow to Generate a Specifier for Use in an Immunoassay

Using established methods and protocols, a monoclonal antibody of IgG isotype with desired specificity and target-binding properties was recombinantly produced using hybridoma cell or a transformed mammalian host cell, wherein the antibody producing cell secretes the antibody into the supernatant. Different antibodies were produced, wherein the antibodies were of human, murine, sheep or rabbit origin. In each case, the respective antibody was purified from the supernatant using chromatographic techniques and fractionation.

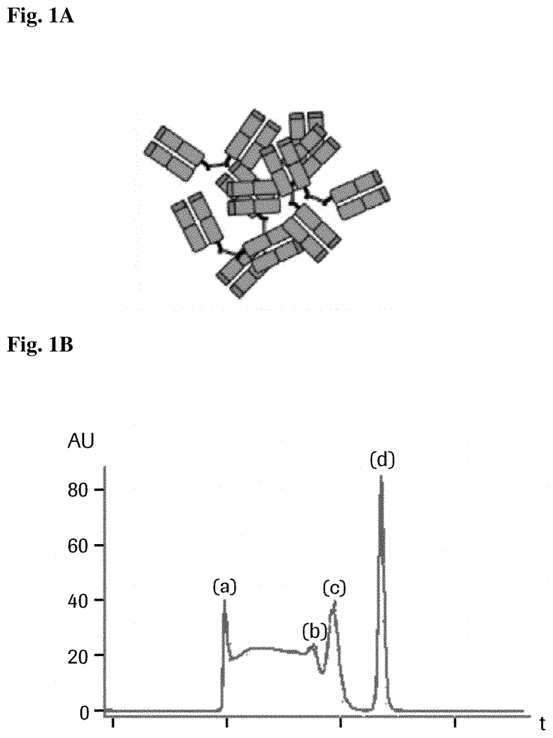

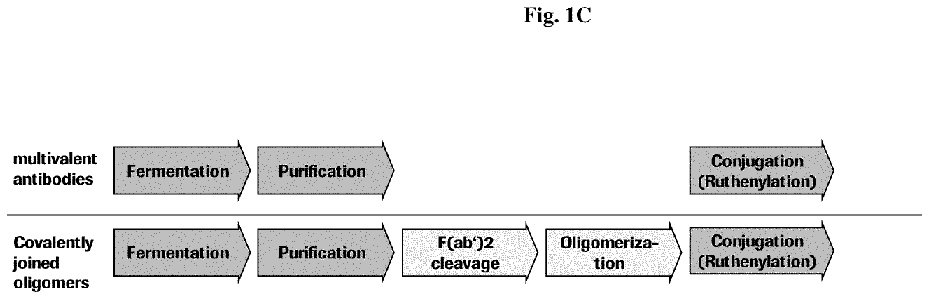

In an embodiment the purified IgG was subjected to enzymatic cleavage to generate Fab fragments or F(ab′)2 fragments. The F(ab′)2 fragments were purified and thereby separated from the Fc parts. Purified F(ab′)2 fragments were cross-linked chemically to form a mixture of oligomers with different molecular weights. FIG. 1 A depicts such an oligomer illustrating the randomness with which the F(ab)2 were combined in the chemical conjugation process of cross-linking Using chromatographic separation techniques the mixture was fractioned and fractions containing F(ab′)2 oligomers of desired size were pooled.

In another embodiment the purified IgG was subjected to enzymatic cleavage to generate Fab fragments. The Fab fragments were purified and thereby separated from the Fc parts. Purified Fab fragments were cross-linked chemically to form a mixture of oligomers with different molecular weights. Using chromatographic separation techniques the mixture was fractioned and fractions containing Fab oligomers of desired size were pooled.

Optionally, IgG molecules were oligomerized without prior enzymatic cleavage.

FIG. 1 B shows a chromatograph representing the outcome of an exemplary cross-linking experiment using F(ab′)2 fragments to generate oligomers, schematically depicted in FIG. 1 A. It is important to appreciate that in FIG. 1 B the area between the peaks designated (a) and (b) represents oligomers of different sizes.

Practically, these are collected as fractions and fractions are separately tested and characterized regarding its suitability of being labeled and used in an immunoassay. For this purpose, the workflow to which the oligomers are subjected to is analogous to the workflow described for multivalent recombinant antibodies in Example 8. That is to say, the heterogeneous mixture of oligomers formed was fractionated by size, and samples of each size fraction were labeled to different average label densities per oligomer. Subsequently, the signal-to-noise ratio was determined for each labeled oligomer sample, and the best-performing samples (i.e. those with highest signal-to-noise ratio) were selected. Oligomer size fractions corresponding to the selected samples were labeled at a density according to the values found for the respective samples that were determined as optimal.

Purified IgG, F(ab′)2 or Fab oligomers of desired sizes were conjugated with detectable label; typically, Ruthenium-based labels were used to generate detection reagents. Labeled oligomers of desired size range as described above were used in immunoassays, wherein the detection step of an immunoassay was performed by generating a signal by way of electrochemiluminescence (ECL).

### Example 3

Expression Vector for the Production of Multivalent IgG-Derived Antibodies

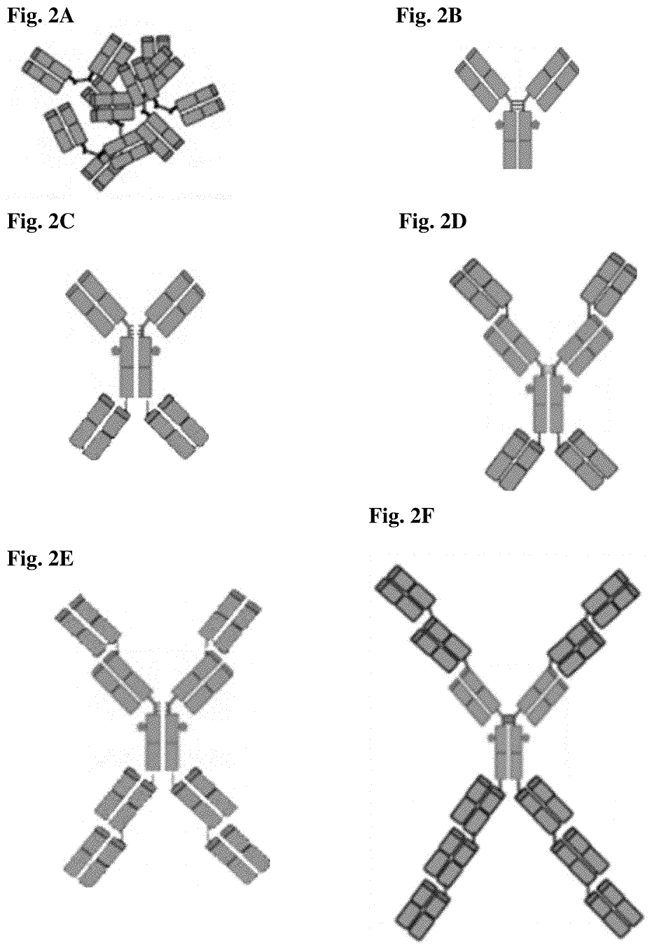

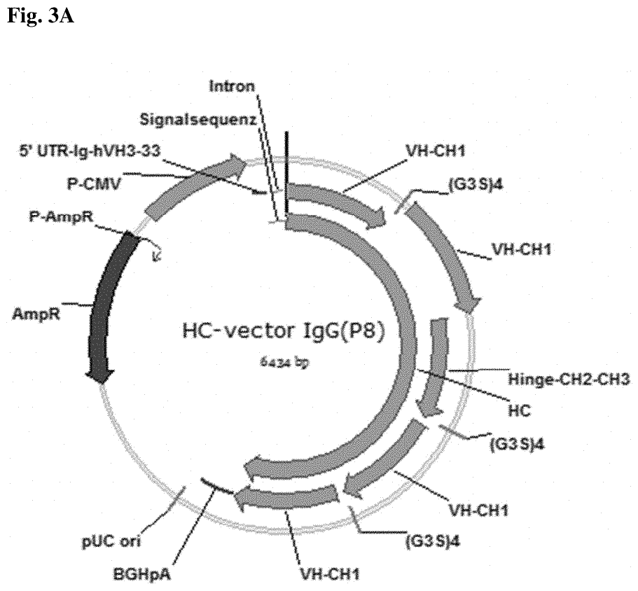

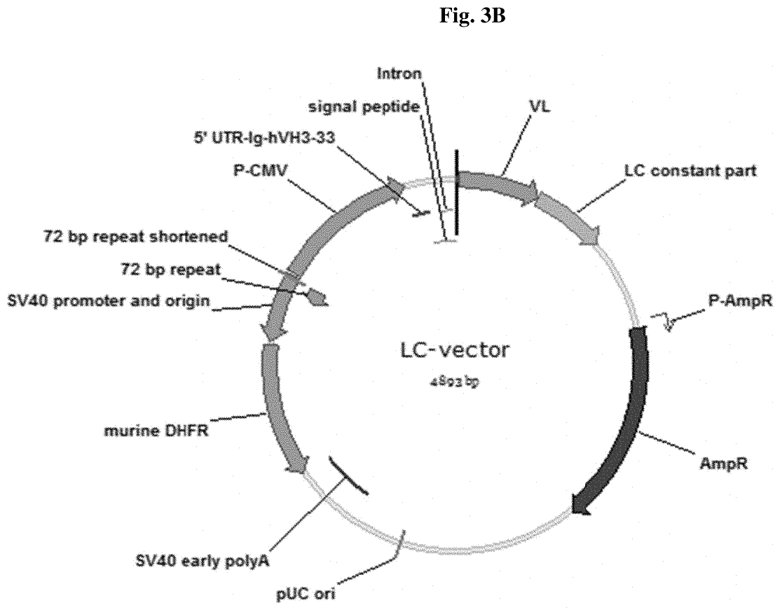

Exemplified is an expression construct for an octavalent IgG(P8) antibody as shown in FIG. 2 E. As shown in FIG. 3 A, a plurality of VH-CH1 sequences flanked by linker sequences (e.g. (G₃S)₄) were added upstream and downstream of Hinge-CH2-CH3 encoding sequences, thereby generating heavy chains encoding for several VH-CH1 domains. In the vector map the heavy chain coding sequence is depicted twice, firstly as a contiguously drawn arrow, and secondly as a composite of several arrows, each representing a modular building block of the whole heavy chain.

The vector of FIG. 3 A is co-expressed with the vector of FIG. 3 B. This light chain expression vector expresses a standard light chain consisting of a VL and a constant domain (kappa or lambda).

FIGS. 3 A and B thus depict examples of heavy and light chain vectors. Building blocks as indicated in Formula I can be appended as indicated for those that were used to express an IgG(P8).

Similar constructs were made to co-express vectors for heavy chains with the corresponding light chains, wherein the vectors for heavy chains encoded different structures of Formula I

_{N-terminus}[Fab_{H}-L-]ₙFab_{H}-L-dd(Fc_{H})[-L-Fab_{H}]_{m C-terminus} (Formula I)

wherein
(a) each of m and n was selected independently from an integer of 1 to 5,
each of m and n was selected such that the value of (2+2*(n+m)) was a value selected from the group consisting of 6, 8, 10, and 12;
(b) “-” was a covalent bond within a polypeptide chain;
(c) each L was optional and, if present, was an independently selected variable linker amino acid sequence, specifically but not exclusively the linker amino acid sequence (G₃S)₄;
(d) Fc_{H} was a heavy chain of a non-antigen binding immunoglobulin region comprising a N-terminal hinge domain; and
(e) each Fab_{H} was independently selected from A_{H} and B_{H}, wherein A_{H} and B_{H} were different, and A_{H} and B_{H} were independently selected from the group consisting of

_{N-terminus}[VH-CH1]_{H C-terminus} (Formula II),

_{N-terminus}[VH-CL]_{H C-terminus} (Formula III),

_{N-terminus}[VL-CL]_{H C-terminus} (Formula IV), and

_{N-terminus}[VL-CH1]_{H C-terminus} (Formula V),
wherein
VH was a N-terminal immunoglobulin heavy chain variable domain,
VL was a N-terminal immunoglobulin light chain variable domain,
CH1 was a C-terminal immunoglobulin heavy chain constant domain 1, and
CL was a C-terminal immunoglobulin light chain constant domain.

The vectors for the expression corresponding light chains encoded Fab_{L} polypeptides

wherein
(a) “-” was a covalent bond within a polypeptide chain; and
(b) each Fab_{L} was independently selected from A_{L} and B_{L}, wherein A_{L} and B_{L} were different, and A_{L} and B_{L} were independently selected from the group consisting of

_{N-terminus}[VH-CH1]_{L C-terminus} (Formula VI),

_{N-terminus}[VH-CL]_{L C-terminus} (Formula VII),

_{N-terminus}[VL-CL]_{L C-terminus} (Formula VIII), and

_{N-terminus}[VL-CH1]_{L C-terminus} (Formula IX).

Importantly, each antigen binding site Fab_{H}:Fab_{L} of the antibody had to be an aligned pair (the alignment being signified by “:”). So the Fab_{H} and Fab_{L} sequences to be co-expressed in a transformed host cell were selected that the Fab_{H} and Fab_{L} portions could form aligned pairs, and each aligned pair was independently selected from the group consisting of A_{H}:A_{L} and B_{H}:B_{L}, wherein A_{H}:A_{L} and B_{H}:B_{L} were selected independently from the group consisting of

[VL-CH1]_{H}:[VH-CL]_{L},

[VL-CL]_{H}:[VH-CH1]_{L},

[VH-CH1]_{H}:[VL-CL]_{L}, and

[VH-CL]_{H}:[VL-CH1]_{L}.

Variations of the above were made, too. For example, the Fc_{H} higher order element in the heavy chain was shortened to a CH3 element, only. Also, one or more Fab_{H} originating from a different species than Fc_{H} were combined in a heavy chain expression vector, thus encoding a chimaeric heavy chain. In most cases, the vector for a expression of light chain polypeptides comprised one Fab_{L} coding sequence. However, light chain vectors with two different light chain expression cassettes were designed, too.

### Example 4

Recombinant Expression and Purification of Multivalent Recombinant Anti-TSH Antibodies

Multivalent monospecific anti-TSH antibodies (TSH=human thyroid-stimulating hormone) were produced recombinantly. For purposes of systematic comparison, the anti-TSH binding sites (A_{H}:A_{L}) were provided using different designs of multivalent recombinant antibodies, as depicted in FIGS. 2 C, D, E and F, and designated IgG(P4), IgG(P6), IgG(P8) and IgG(P12), respectively.

Recombinant expression was performed transiently in human embryonic kidney (HEK) cells, or transiently or non-transiently in CHO cells. Transformed cells secreted the multivalent monospecific anti-TSH antibodies into the serum-free culure supernatant from which they were isolated.

Similar to the original bivalent anti-TSH monoclonal antibody, the multivalent anti-TSH antibodies could be produced with sufficient yields and without significant losses during purification using protein A affinity chromatography of culture supernatant. Alternatively, the multivalent recombinant antibodies were purified using ion exchange chromatography (IEX) to which the culture supernatant was subjected.

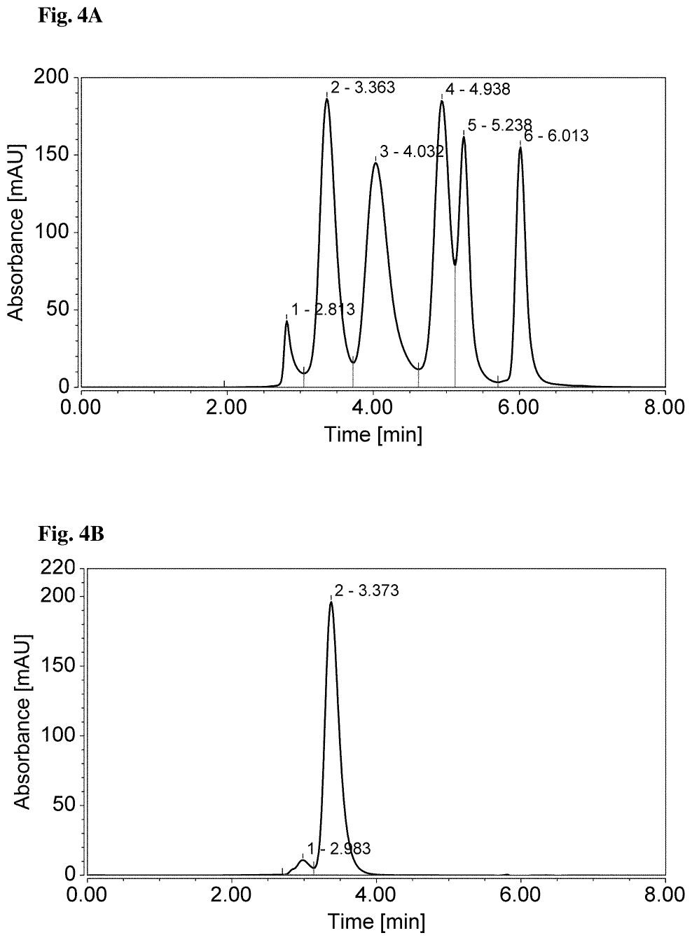

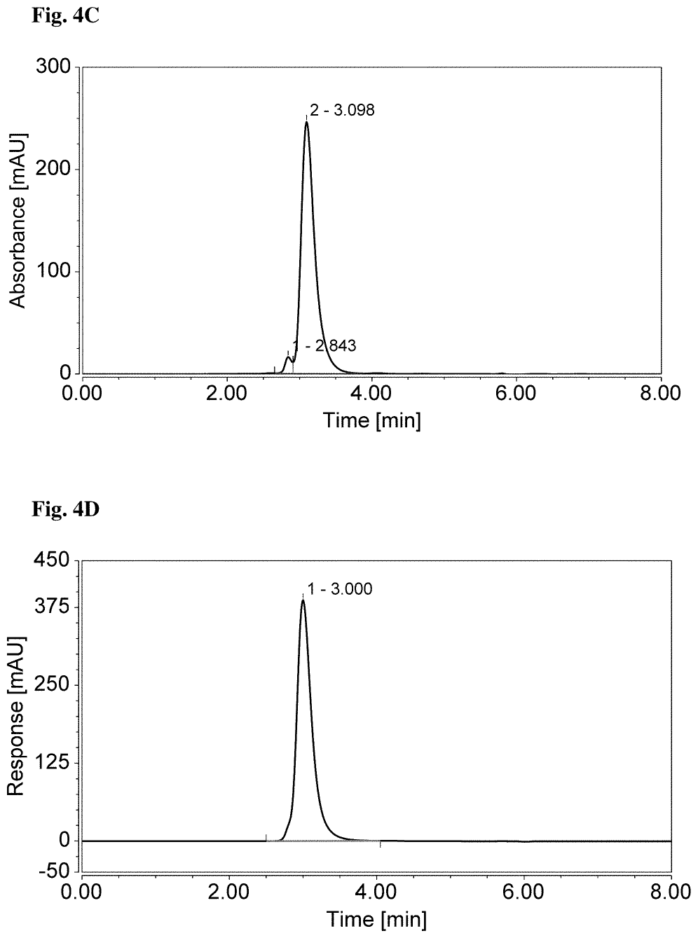

The percentage of aggregates that were observed in all antibody formats tested was always less than 5% of total antibody protein. An aggregate-related peak can be seen as the small shoulder to the left of the respective main peak, in FIG. 4 B, C.

Table 1 shows expression yields and amounts of aggregates observed by way of GFC300 or TSK4000 gel filtration (following chromatographic purification as indicated). For details of gel filtration also see Example 5. The IgG reference given in Table 1 reflects the data obtained for the original bivalent anti-TSH monoclonal antibody (TSH=thyroid-stimulating hormone).

TABLE 1

| MAB<TSH> | IgG | | | | |
|---|---|---|---|---|---|
| CLONE 1 | reference | IgG(P4) | IgG(P6) | IgG(P8) | IgG(P12) |
| Expression | 13.8 mg | 13.8 mg | 6.2 mg | 8.7 mg | 2.3 mg |
| yield | | | | | |
| (per 500 ml | | | | | |
| culture | | | | | |
| supernatant) | | | | | |
| Chroma- | Protein | Protein | Protein | Protein | Protein |
| tographic | A | A | A | A | A |
| purification | | | | | |
| material | | | | | |
| Aggregates | <2% | <5% | <2% | <2% | <2%* |
| (GFC300; | | | | | |
| *TSK4000) | | | | | |

### Example 5

Analytics of Purified Multivalent Recombinant Antibodies

Multivalent monospecific anti-TSH antibodies were produced and purified as in Example 4 described above. Preparation/isolation using Protein A was performed with all antibodies tested. The isolated bivalent and multivalent monospecific anti-TSH antibodies were subjected to analytical size exclusion chromatography (SEC). Further, isolated bivalent and multivalent monospecific anti-TSH antibodies were subjected to SDS-PAGE. In each case, IgG anti-TSH monoclonal antibodies were isolated likewise and used as a reference in analytical experiments.

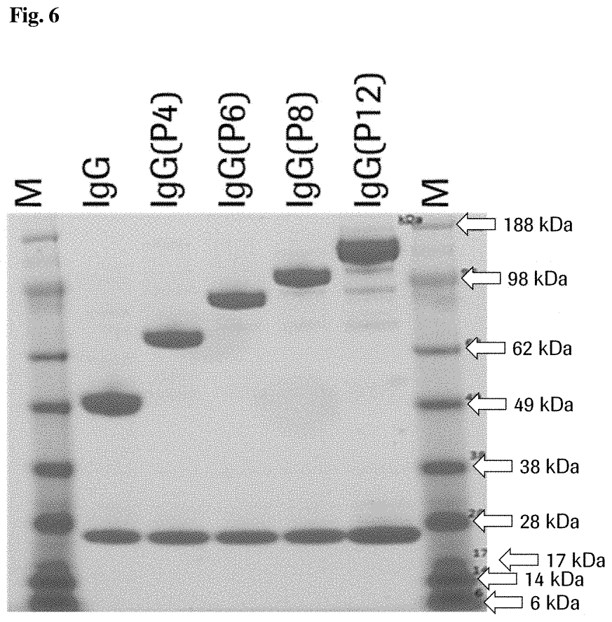

FIG. 6 shows the results of PAGE relative to size marker proteins. Notably, the heavy chains of different sizes can be recognized, as well as the differing amounts of light chains, visible as band strengths. The gel also indicates the purity of the preparations.

FIGS. 4 A, B, C and D show the results of size exclusion chromatography (SEC) experiments with TSKgel QC-PAK GFC 300 (Tosoh) chromatographic material. FIG. 4 A depicts the result of a calibration standard (markers for different molecular weights), wherein the six peaks represent the following markers (from left to right): dimers of beta-galactosidase, beta-galactosidase (465 kDa), sheep IgG (150 kDa), Sheep Fab (50 kDa), myosin light chain (17 kDa), Glycine-Tyrosine dipeptide (233 Da).

With regards to the multivalent recombinant forms of MAB<TSH>CLONE 1, FIGS. 4 B, C, and D show the results for IgG(P4), IgG(P6), and IgG(P8), respectively. In FIGS. 4 B and C the small peak to the left of the main peak was interpreted to represent low amounts of aggregates of the respective recombinant multivalent antibody. Remarkably, the extra peak is missing in FIG. 4 D indicating that aggregates were detectably absent in this preparation using TSKgel QC-PAK GFC 300 (Tosoh) chromatographic material.

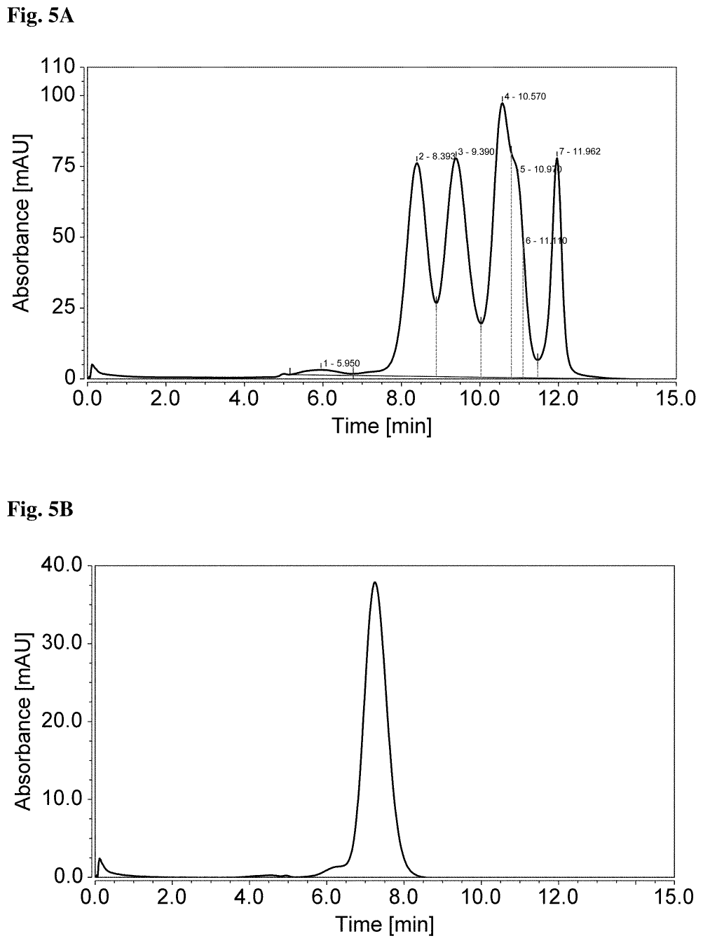

The same experiments were repeated with different chromatography materials. FIGS. 5 A and B show results obtained using TSKgel G4000SWx1 (Tosoh) chromatographic material. FIG. 5 A depicts the results for the same standards, dimers of beta-galactosidase, beta-galactosidase (465 kDa), sheep IgG (150 kDa), Sheep Fab (50 kDa), myosin light chain (17 kDa), Glycine-Tyrosine dipeptide (233 Da). The peaks that were generated by sheep IgG (150 kDa), Sheep Fab (50 kDa) were not resolved as clearly separate peaks but resulted in a broad peak with a shoulder to the right corresponding to the Fab.

FIG. 5 B shows the results for MAB<TSH>CLONE 1 IgG(P8). The sholder on the left of the main peak is an indication of the presence of aggregates, however at very low amounts.

In summary it was concluded that recombinantly expressed multivalent monospecific antibodies could be produced and purified without extensive effort and with high purity.

### Example 6

Characterization of Target Binding Kinetics

Multivalent monospecific anti-TSH antibodies were produced and purified as in Example 4 described above. The kinetic analysis was performed at 37° C. on a GE Healthcare Biacore 4000 instrument. A Biacore CMS series S sensor was mounted into the instrument and was hydrodynamically addressed and preconditioned according to the manufacturer's instructions. The system buffer was HBS-EP (10 mM HEPES (pH 7.4), 150 mM NaCl, 1 mM EDTA, 0.05% (w/v) P20). The sample buffer was the system buffer supplemented with 1 mg/ml CMD (Carboxymethyldextran, Fluka).

The following capture system was established on the biosensor. A monoclonal anti-human IgG Fc capture antibody was immobilized according to the manufacturer's instructions using NHS/EDC chemistry. The sensor was subsequently saturated with 1 M ethanolamine pH 8.5. Immobilized antibodies were saturated with the respective recombinant multivalent antibody. Different capture spots were used for the interaction measurements and for references. Recombinantly produced TSH target antigen was diluted at different ratios in sample buffer and was injected at a flow rate of 30 μl/min for 1 min. The recombinant antibody Capture Level (CL) in response units (RU) was monitored.

Table 3 represents the results obtained for different multivalent recombinant antibodies, and IgG for comparison. KD indicates the equilibrium dissociation constant between the antibody and the antigen, and its value is expressed in [nM]. The parameter kₒₙ reflects the association rate expressed as [1/Ms], and K_{off} the dissociation rate, expressed as [1/s]. The parameter t/2_{diss} describes the half-time of analyte bound to the antibody, expressed in [min]. The ratio of the molar amount of antigen bound by a given molar amount of a multivalent recombinant antibody is expressed as AG/AB.

TABLE 3

| MAB<TSH> | IgG | | | | |
|---|---|---|---|---|---|
| CLONE 1 | reference | IgG(P4) | IgG(P6) | IgG(P8) | IgG(P12) |
| KD at 25° C. | 0.22 | 0.21 | 0.25 | 0.18 | 0.23 |
| KD at 37° C. | 0.49 | 0.64 | 0.61 | 0.65 | 0.71 |
| kₒₙ | 4.9 E+05 | 3.3 E+05 | 3.6 E+05 | 3.5 E+05 | 3.4 E+05 |
| t/2_{diss} | 108 | 168 | 133 | 183 | 150 |
| molar ratio | 1.9 | 3.8 | 5.8 | 7.6 | 12.0 |
| (AG/AB) | | | | | |

For “E+05” read: multiplied with 10⁵, in line with the general understanding of the skilled person

Remarkably, the molar ratios that were found correlated very closely with the amount of antigen binding sites of the respective antibody. It can be concluded from this result that the multivalent recombinant antibodies as described herein actually do provide as many functional antigen binding sites as laid out in their design. Thus, the design allow to put together multivalent target binding functions (antigen binding sites) simply and reproducibly, and actually reflecting predictions based on molecular design. This is in stark contrast with the multivalent oligomers that are obtained using so far established techniques (see Example 2).

### Example 7

Labeling of Multivalent Recombinant Antibodies

Ruthenium conjugates were generated with increasing label incorporation. Purified multivalent recombinant antibodies were used in labeling experiments. Recombinant antibodies were as depicted in FIGS. 2 C, D, E and F, and designated IgG(P4), IgG(P6), IgG(P8) and IgG(P12), respectively. In labeling experiments different amounts of the respective antibody were reacted with either the labeling reagent tris-bipyridyl-ruthenium or its sulfonated form (also referred to as “sBPRu”) using standard NHS ester coupling chemistry. Under these conditions Ruthenium label is covalently attached to functional groups of Lysine amino acid residues in the antibody backbone chain.

The average incorporation of Ruthenium label can be determined as the number of protein-bound label molecules per antibody. It can be measured quantitatively by separately determining in a sample of labeled antibody the amount of protein and the amount of Ruthenium label. Exemplary methodological approaches are mass spectrometry of photometric determinations.

Table 4 represents a comparison of different multivalent recombinant antibodies and IgG, with respect to the incorporation rate of sBPRu.

TABLE 4

| Antibody | IgG | | | | |
|---|---|---|---|---|---|
| architecture/design | reference | IgG(P4) | IgG(P6) | IgG(P8) | IgG(P12) |
| Incorporation of | 1-25.2 | 1-21.4 | 1-43.6 | 1-47.3 | 1-23.2 |
| exemplary sBPRu | | | | | |
| (range of average | | | | | |
| label density, with | | | | | |
| observed upper limit) | | | | | |
| Number of | 8 | 9 | 14 | 17 | 3 |
| conjugation reactions | | | | | |
| independently tested | | | | | |

Surprising was the finding that specifically P6 and P8 antibodies showed especially high values. Thus, P6 and P8 allow for especially efficient labeling.

Similar results were observed with other labeling reactions with other labels using NHS ester (N-hydroxy-succinimide) coupling chemistry.

### Example 8

Electrochemiluminescence Signal Counts Generated by Ruthenium-Labeled Antibodies

Multivalent monospecific anti-TSH antibodies were produced and purified as in Example 4 described above.

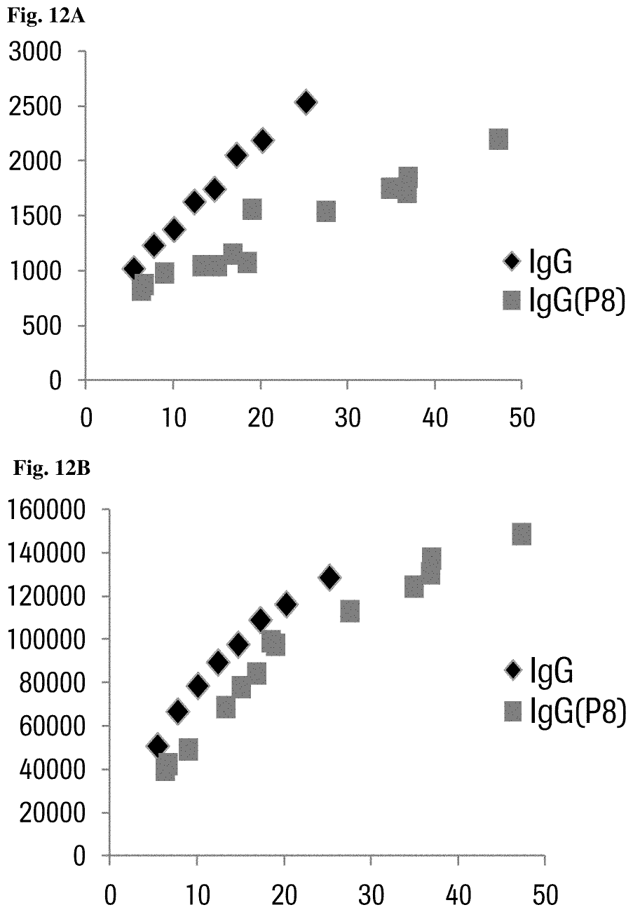

The anti-TSH Roche Cobas Elecsys assay of Roche catalogue number 07028091190 was performed in variations as described. Only the Ruthenium-labeled detection agent (Ru-labeled oligomeric antibody fragments) of the Cobas assay was replaced by a recombinant multimeric antibody with a specific density of Ruthenium label determined previously. Similarly, IgG was used as a reference. Exemplary results for IgG and IgG(P8) are depicted in FIG. 12 A for blank calibration measurements (no target antigen present, null measurements), and in FIG. 12 B for measurements with a target antigen concentration of 5 μU TSH/ml. In each diagram the ordinate represents electrochemiluminescent signal strength, i.e. Ruthenium light counts detected by the Elecsys instrument; the abscissa represents the average density of incorporated Ruthenium label per antibody.

The data show that in the experiments with the blanks (no TSH antigen present) the signal increase as a function of increased label density is higher for IgG, compared with the multivalent construct IgG(P8). The signal obtained with the blanks is also referred to as “noise”.

A similar finding is observed when the TSH target is measured at a concentration of 5 μU TSH/ml. A value measured on the basis of an actually present antigen is referred to as “signal”.

It was found that an IgG(P8) and an IgG can produce the same noise or signal, whereby under the conditions tested the IgG(P8) always had a higher label density than the IgG which produced an about equally high amount of light electrochemiluminescent light counts.

This finding was interpreted to indicate that labeled IgG(P8) are capable of generating a technically more favourable signal-to-noise ratio, in contrast to IgG.

Table 5 represents the measurement values obtained for IgG, which are the basis for the graphs in FIG. 12A.

TABLE 5

| A | | | | |
|---|---|---|---|---|
| Ruthenium | 5.5 | 7.8 | 10.1 | 12.4 |
| incorporation | | | | |
| 5 μU | 50903 | 66748 | 78462.5 | 89122.5 |
| TSH per ml | | | | |
| 0 μU | 1016 | 1231.5 | 1372.5 | 1629.5 |
| TSH per ml | | | | |
| S/N | 50.10 | 54.20 | 57.17 | 54.69 |
| B | | | | |
| Ruthenium | 14.8 | 17.3 | 20.3 | 25.2 |
| incorporation | | | | |
| 5 μU | 97682.5 | 108786.5 | 116232 | 128403.5 |
| TSH per ml | | | | |
| 0 μU | 1739 | 2053 | 2186.5 | 2540 |
| TSH per ml | | | | |
| S/N | 56.17 | 52.99 | 53.16 | 50.55 |

S/N: signal-to-noise ratio
Ruthenium incorporation denotes average amount of Ru label per antibody

Table 6 represents the measurement values obtained for IgG(P8), which are the basis for the graphs in FIG. 12 B.

TABLE 6

| A | | | | |
|---|---|---|---|---|
| Ruthenium | 6.4 | 6.7 | 9 | 13.3 |
| incorporation | | | | |
| 5 μU | 39750 | 42204 | 49395.5 | 68622.5 |
| TSH per ml | | | | |
| 0 μU | 829 | 875.5 | 975.5 | 1049 |
| TSH per ml | | | | |
| S/N | 47.95 | 48.21 | 50.64 | 65.42 |
| B | | | | |
| Ruthenium | 15.1 | 16.8 | 18.5 | 19.1 |
| incorporation | | | | |
| 5 μU | 78222.5 | 84320.5 | 99169.5 | 97534 |
| TSH per ml | | | | |
| 0 μU | 1043.5 | 1154.5 | 1077.5 | 1558.5 |
| TSH per ml | | | | |
| S/N | 74.96 | 73.04 | 92.04 | 62.58 |
| C | | | | |
| Ruthenium | 27.5 | 35 | 36.8 | 36.9 |
| incorporation | | | | |
| 5 μU | 113146.5 | 124616 | 130570.5 | 137163.5 |
| TSH per ml | | | | |
| 0 μU | 1545 | 1751 | 1719 | 1845.5 |
| TSH per ml | | | | |
| S/N | 73.23 | 71.17 | 75.96 | 74.32 |
| D | | | | |
| Ruthenium | 47.3 | | | |
| incorporation | | | | |
| 5 μU | 148618 | | | |
| TSH per ml | | | | |
| 0 μU | 2194 | | | |
| TSH per ml | | | | |
| S/N | 67.74 | | | |

S/N: signal-to-noise ratio
Ruthenium incorporation denotes average amount of Ru label per antibody

### Example 9

Determining Optimal Label Density Per Recombinant Antibody to Obtain Optimal Signal-To-Noise Ratio in Immunoassays

Multivalent monospecific anti-TSH antibodies were produced and purified as in Example 4 described above.

Firstly, an amount of calibrator TSH antigen (5 μU TSH/ml, as determined by the Roche Cobas Elecsys assay of Roche catalogue number 07028091190, Roche Diagnostics GmbH, Mannheim, Germany) was provided. Also provided were anti-TSH multivalent recombinant antibodies as depicted in FIGS. 2 C, D, E and F, designated IgG(P4), IgG(P6), IgG(P8) and IgG(P12), respectively. As a reference, TSH-specific IgG was provided. Importantly, each antibody was provided in different samples, wherein the samples differed with regards to the average label density per respective antibody.

In a series of experiments as described in Example 8, each antibody with a specific pre-determined average label density was used in Elecsys runs and signal counts corresponding to 5 μU TSH/ml were recorded. Each measured signal-to-noise value for a labeled antibody was subsequently normalized against the corresponding value determined using the original anti-TSH Roche Cobas Elecsys assay of Roche catalogue number 07028091190.

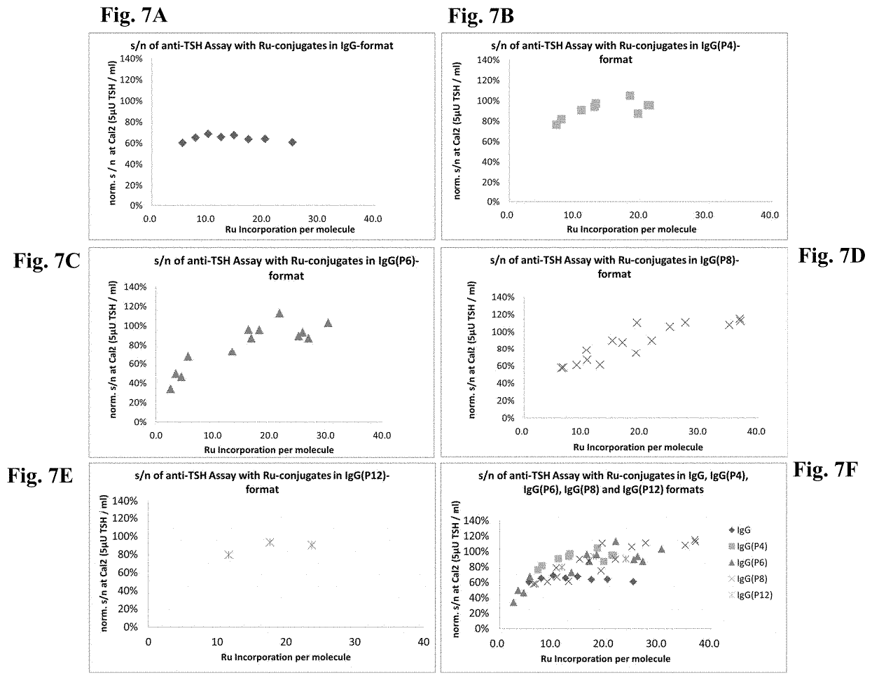

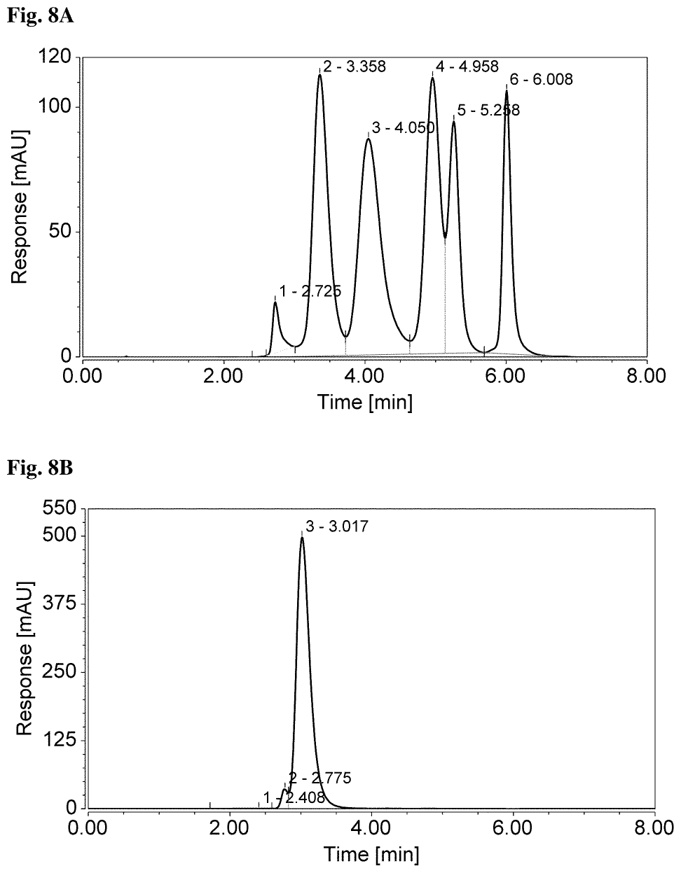

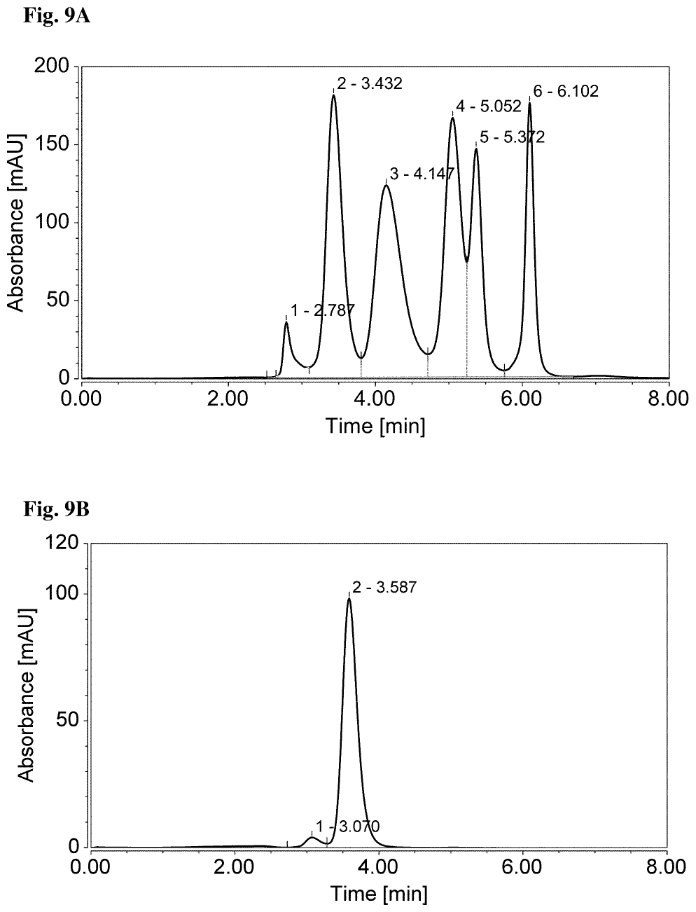

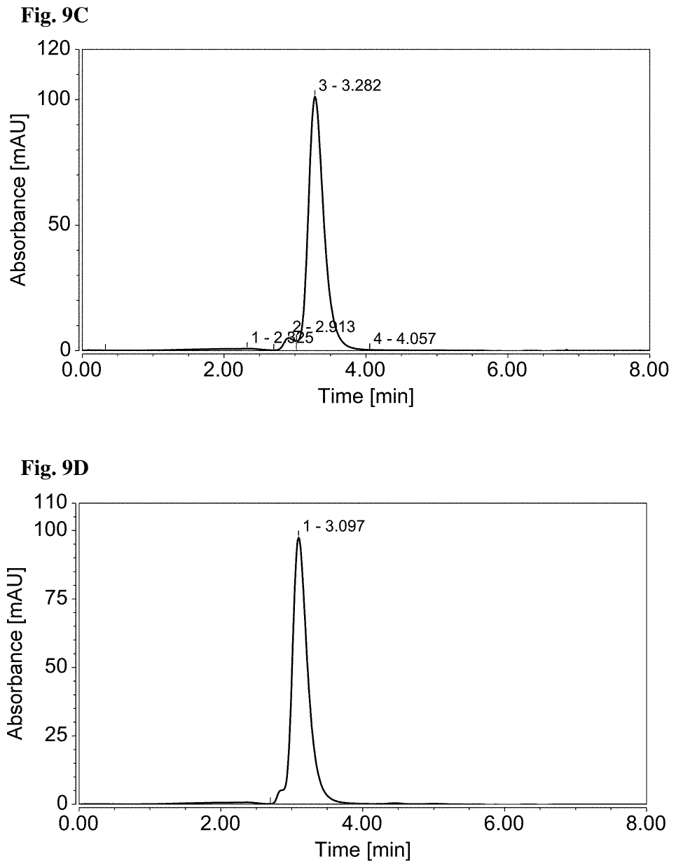

Accordingly, each of the diagrams in FIGS. 7 A to F illustrating the results comprises an ordinate with a scale indicating percentages with the 100% mark corresponding to the measurement obtained with the original anti-TSH Roche Cobas Elecsys assay. Each measured value normalized in this fashion against the 100% reference value of the original assay provides an indication for the detection capability of the respective labeled antibody with which the normalized value was generated. If the normalized value is below 100%, the respective antibody with the given label density is technically less preferred; on the other hand, a normalized value above 100% represents an antibody with a more favourable signal-to-noise ratio which outperforms the labeled oligomers of the original assay.

It is important to appreciate that in the experiments with the labeled antibodies, these were the only component that was changed in the original assay, replacing the labeled oligomers of chemically linked antibody fragments.

In FIG. 7, A depicts the results obtained for IgG, B depicts the results obtained for IgG(P4), C depicts the results obtained for IgG(P6), D depicts the results obtained for IgG(P8), and E depicts the results obtained for IgG(P12). FIG. 7 F depicts the overlay of A through E, thus combining all results. It became apparent that specifically IgG(P6) and IgG(P8) with certain loads of label were capable of outperforming the original assay. Thus, the average preferred label densities as determined using the normalized data allowed to define best-performing labeled conjugates suitable for use in desired immunoassays, specifically diagnostic immunoassays. Those recombinant multivalent antibodies with label densities leading to the highest relative values were chosen for further experimentation. The same was made concerning labeled IgG.

In this regard it is noted that the process of identifying optimal label densities as described above is also performed for each newly synthesized batch of chemically linked antibodies or fragments thereof, as described in Example 2. That is to say, size fractions of oligomers are routinely labeled to different densities, in order to find out which combination of oligomer size and label density is capable of producing equivalent results as the original assay.

Hence, the selection process to determine optimal label density, in its generic way, represents an already established standard practice in the art.

### Example 10

Evaluation of Multivalent Anti-TSH Antibodies for the Detection of Different Concentrations of TSH Antigen, and Comparison with Standard Antibody Fragment Oligomers

The original anti-TSH Roche Cobas Elecsys assay of Roche catalogue number 07028091190 comprising chemically linked IgG fragments labeled with Ruthenium as the detection reagent was used to measure a dilution series of TSH antigen, wherein each aliquot containing a dilution of the TSH antigen was prepared in universal diluent, commercially available as Roche catalogue number 1173277122 (Roche Diagnostics GmbH, Mannheim, Germany). The series of TSH concentrations provided by the dilution aliquots was selected to represent the range of physiological concentrations of at least 95% of the patient population.

Subsequently, the detection reagent was replaced by the multivalent anti-TSH antibodies with optimal label density (see Example 9). Tables 7 and 8 summarize the results, wherein signal-to-noise ratios are tabulated as percentage values relative to the standard assay with the original the detection reagent, i.e. comprising the chemically linked antibody fragments labeled with Ruthenium. For the data in Table 8, the measurements were taken after inclusion of an additional pre-wash step. That is to say, before the detection complexes were allowed to proceed into the measurement cell of the Elecsys instrument, the detection complexes were magnetically immobilized and washed with an additional volume of buffer, thereby clearing out undesired components more efficiently. Table 7 presents the data of the measurements without the pre-wash step, therefore leading to somewhat lower signal-to-noise ratios.

TABLE 7

| | Original | | | | | |
|---|---|---|---|---|---|---|
| | detection | | | | | |
| μU TSH | reagent | | IgG | IgG | IgG | IgG |
| per ml | (oligomers) | IgG | (P4) | (P6) | (P8) | (P12) |
| 5 | 100.00% | 74.74% | 98.56% | 110.79% | 111.78% | 91.87% |
| 2.5 | 100.00% | 74.13% | 97.88% | 109.68% | 110.74% | 91.28% |
| 1.25 | 100.00% | 74.27% | 98.60% | 111.42% | 111.00% | 91.37% |
| 0.625 | 100.00% | 76.07% | 99.33% | 108.83% | 111.78% | 93.12% |
| 0.3125 | 100.00% | 77.21% | 98.72% | 108.52% | 112.00% | 93.41% |
| 0.15625 | 100.00% | 80.13% | 100.34% | 108.23% | 109.26% | 93.01% |
| 0.078125 | 100.00% | 82.15% | 98.73% | 107.18% | 105.83% | 93.04% |

Measurements without Pre-Wash

TABLE 8

| | Original | | | | | |
|---|---|---|---|---|---|---|
| | detection | | | | | |
| μU TSH | reagent | | IgG | IgG | IgG | IgG |
| per ml | (oligomers) | IgG | (P4) | (P6) | (P8) | (P12) |
| 5 | 100.00% | 96.34% | 122.68% | 154.65% | 168.22% | 130.78% |
| 2.5 | 100.00% | 97.02% | 124.77% | 156.73% | 168.76% | 131.64% |
| 1.25 | 100.00% | 96.12% | 124.14% | 155.33% | 165.35% | 130.09% |
| 0.625 | 100.00% | 97.27% | 122.86% | 152.89% | 166.58% | 130.39% |
| 0.3125 | 100.00% | 97.05% | 121.01% | 150.52% | 165.26% | 130.86% |
| 0.15625 | 100.00% | 99.12% | 118.15% | 145.16% | 158.23% | 125.05% |
| 0.078125 | 100.00% | 97.45% | 113.17% | 134.38% | 145.01% | 118.31% |

Measurements with Pre-Wash

The signal-to-noise (s/n) values were calculated and normalized with respect to the current commercial TSH Elecsys assay of Roche catalogue number 07028091190. The results indicated that conjugates from multivalent IgG(P6) and IgG(P8) show best results regarding signal-to-noise, with more than 160% better values at high and low TSH concentrations than the original assay that included a standard detection reagent with covalently chemically crosslinked antibody fragments.

### Example 11

Anti-Troponin-T (TN-T) Multivalent Antibody IgG(P8)

Heavy chain and light chain expression vectors for an octavalent antibody were constructed and transiently expressed in HEK293F host cells which secreted the antibody into the serum-free culture supernatant. The antibody was isolated from the supernatant using Protein A affinity chromatography. Purified antibody could be produced with a yield of 61 mg/l supernatant. GFC300 analytical SEC showed that purified multivalent anti-TNT antibodies were pure and showed only a low amount of aggregation.

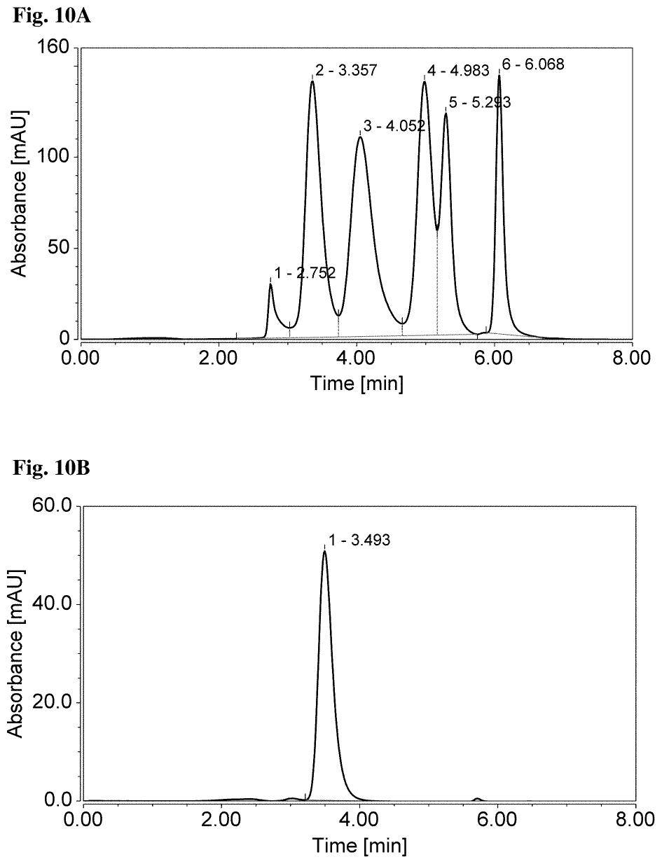

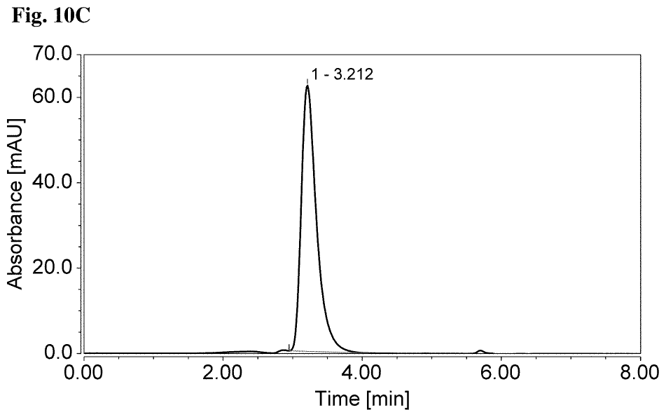

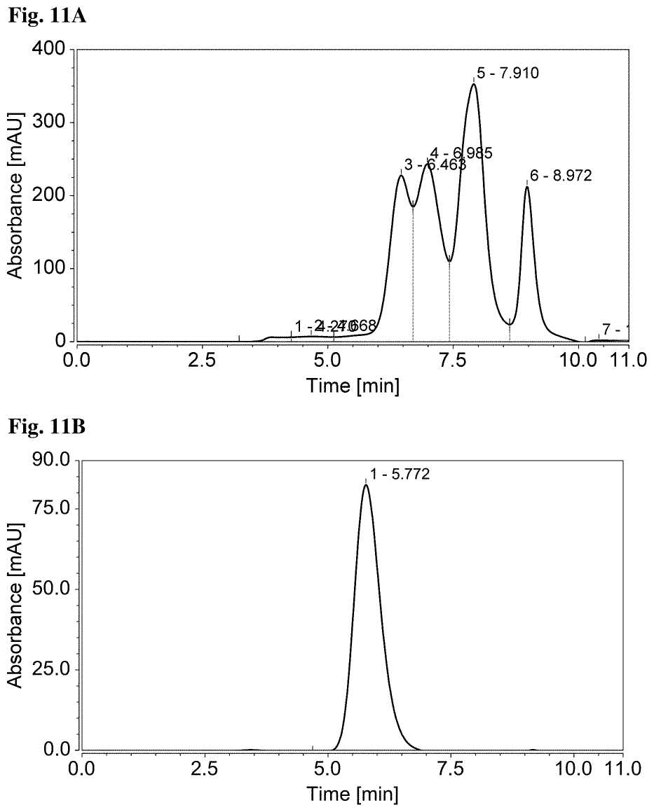

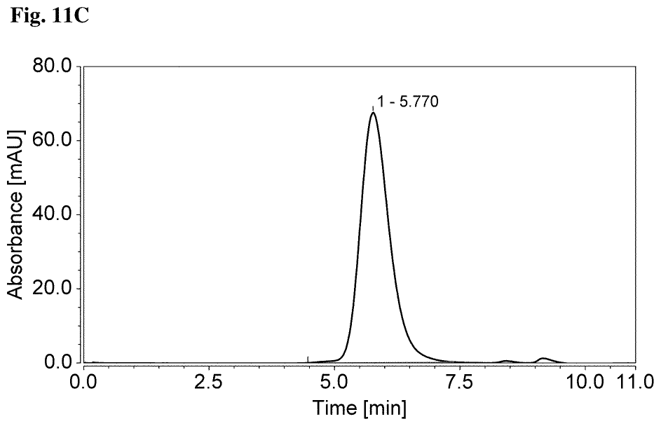

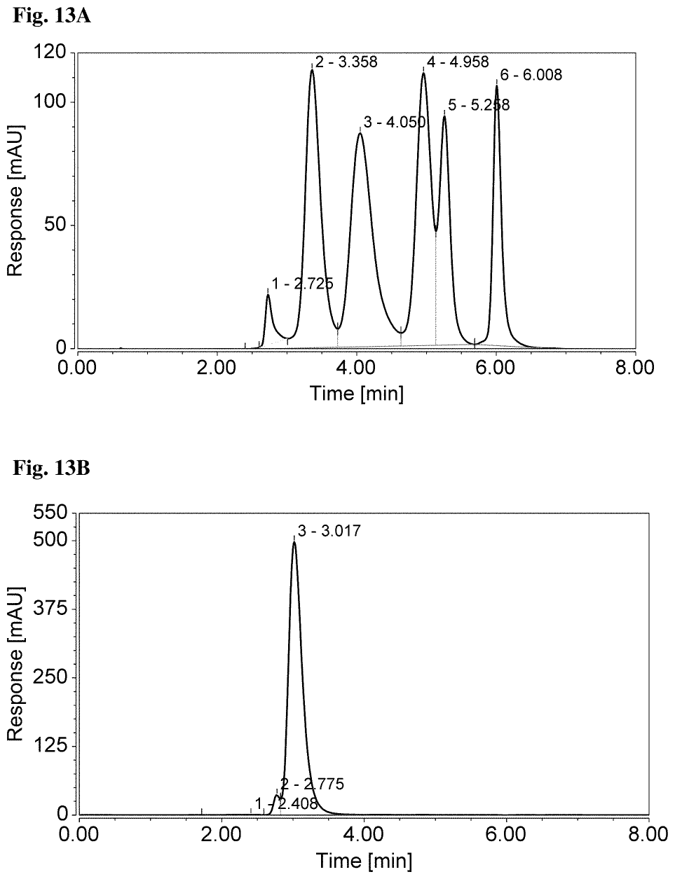

FIG. 13 B shows rhe results of SEC analysis, FIG. 13 A provides the same size standards as shown in FIG. 10 A.

An IgG(P8) Ruthenium conjugate was generated to replace the original standard chemically cross-linked Ru conjugate of the original Roche Elecsys assay, catalogue number 05092744190 (Roche Diagnostics GmbH, Mannheim, Germany). At all tested concentrations of the target antigen TN-T, ranging from 4.5-4000 ng/ml, the IgG(P8)-Ru conjugate showed superior performance in the Elecsys TN-T Assay.

The data in Table 9 were obtained in an analogous way as the data in Example 10. A pre-wash step was included.

TABLE 9

| | Original | |
|---|---|---|
| | detection | |
| ng TN-T | reagent | |
| per ml | (oligomers) | IgG(P8) |
| 4000 | 100.00% | 124.02% |
| 2000 | 100.00% | 113.79% |
| 1000 | 100.00% | 110.48% |
| 500 | 100.00% | 113.32% |
| 100 | 100.00% | 135.78% |
| 50 | 100.00% | 140.54% |
| 25 | 100.00% | 141.82% |
| 18 | 100.00% | 121.23% |
| 9 | 100.00% | 118.72% |
| 4.5 | 100.00% | 109.08% |

Measurements with Pre-Wash

### Example 12

Anti-HIV Antigen Multivalent Antibodies

Finally, these novel multivalent antibody formats were tested in the HIV-antigen assay, catalogue number 11971611122 (Roche Diagnostics GmbH, Mannheim, Germany). This assay uses two separate ruthenylated anti-p24 antibody fragment oligomers. That is to say, the first and the second oligomer are specific for the detection of a first and a second epitope of the p24 target protein. Both anti-p24 monoclonal IgG clones (E and D) were used to construct IgG(P4), IgG(P6) and IgG(P8) formats. Additionally an IgG(P8) variant was generated with four antigen binding sites from clone E and four binding sites from clone D, thus resulting in an octavalent and bispecific antibody. The bispecific property was generated by using human and mouse CH1 and Cₖₐₚₚₐ sequences, respectively. All molecules were expressed in HEK293, and purified via protein A chromatography. Analytical-SEC showed that all multivalent anti-p24 antibodies E, D and the biclonal E/D molecule could be produced with high purity and low amount of aggregation in IgG(P4), IgG(P6) and IgG(P8) formats. All constructs were expressed in HEK293, purified via protein A chromatography. All proteins were conjugated with Ruthenium at the optimal label incorporation rate.

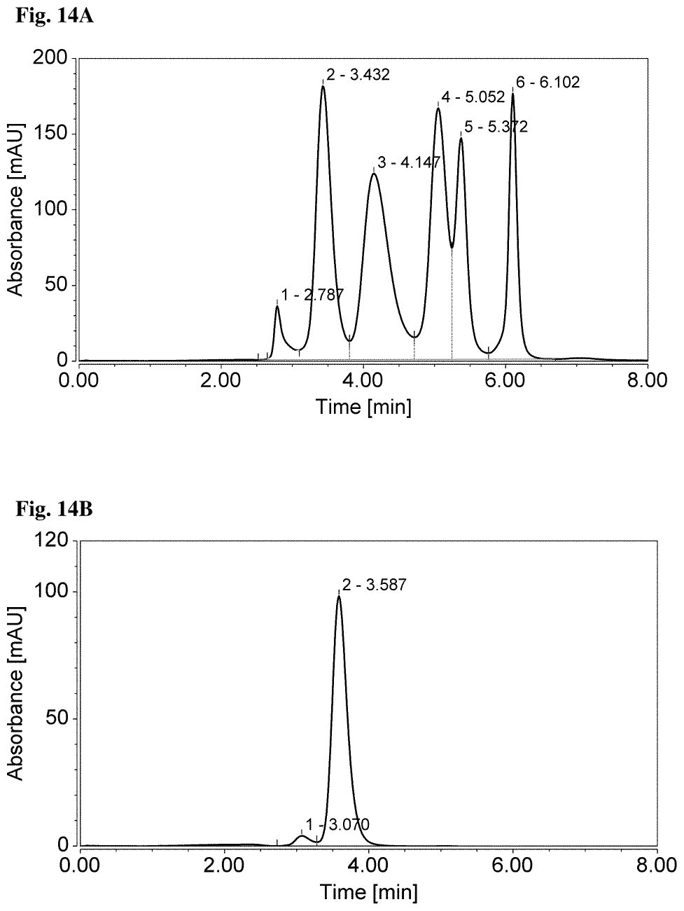

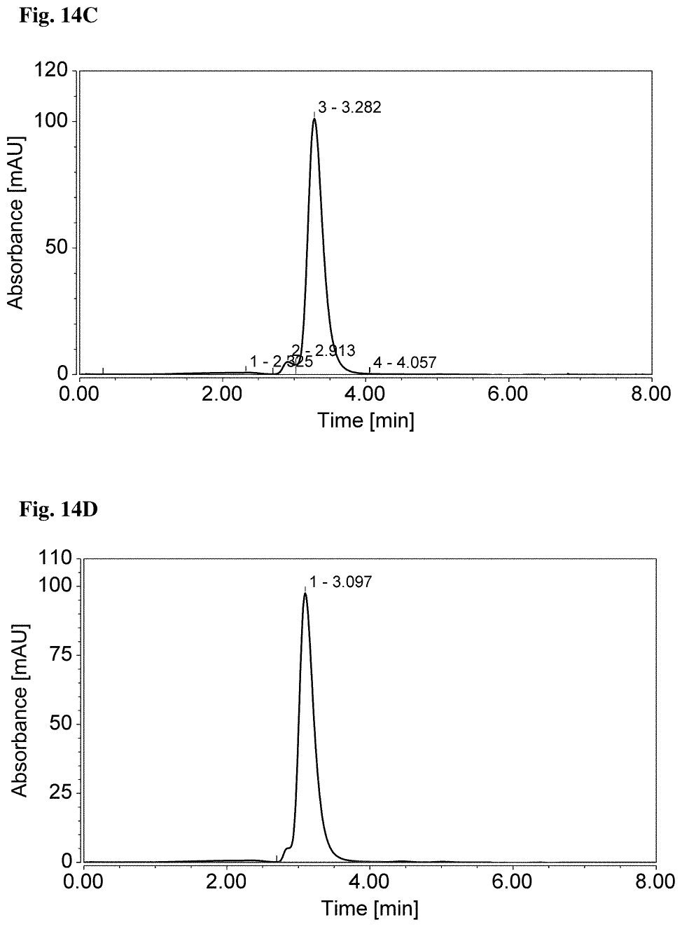

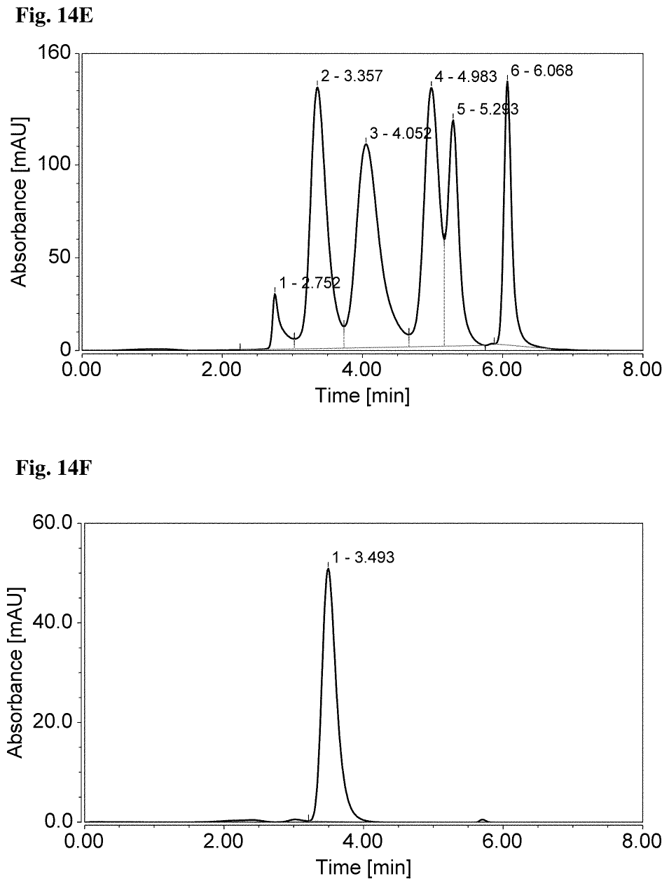

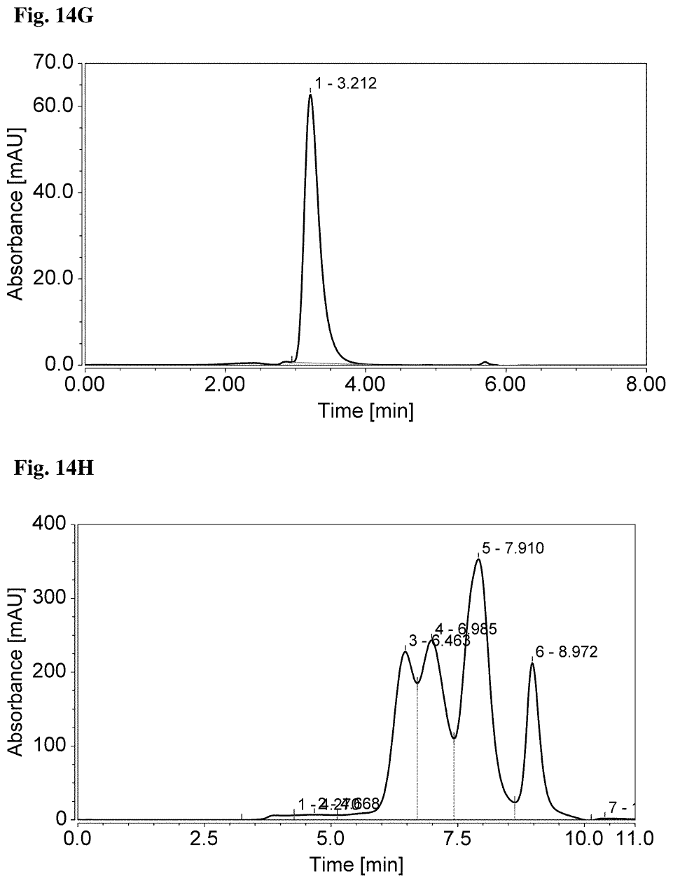

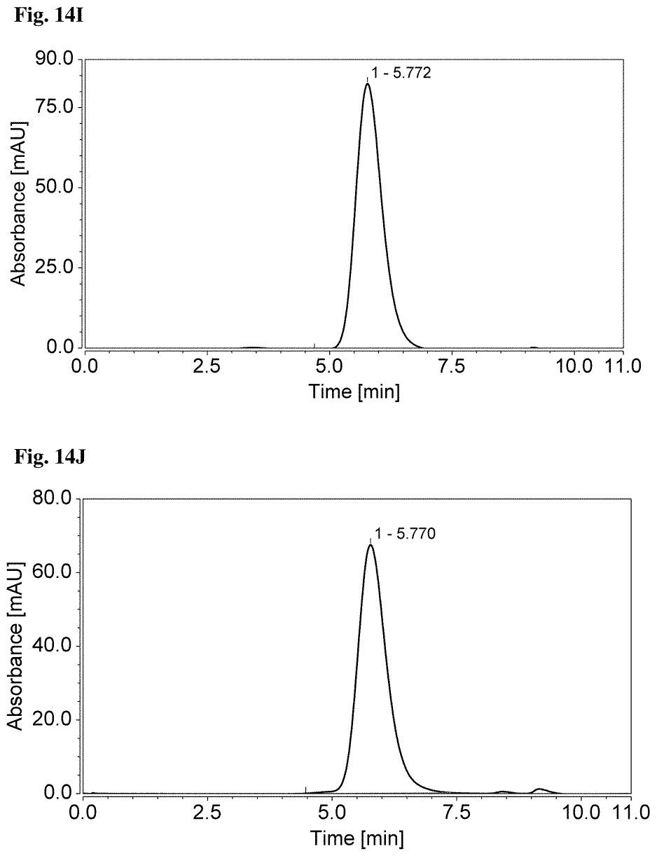

FIG. 14 A shows a size standard analogous to the chromatogram shown in FIG. 10 A; FIGS. 14 B, C, and D show the monospecific antibodies P4, P6, and P8, respectively, for the E specificity. For the D specific constructs FIG. 14 E hows a size standard analogous to the chromatogram shown in FIG. 10 A, FIGS. 14 F and G show the monospecific antibodies P4, P6, respectively for the D specificity.

FIG. 14 H shows a size standard analogous to the chromatogram shown in FIG. 10 A, but with a different SEC material, namely Superose 6. Using the same SEC material the monospecific P8 construct for the D specificity was analyzed, as shown in FIG. 14 I. FIG. 14 J shows the bispecific P8 construct having four E antigen binding sites and four D antigen binding sites, also analyzed using Superose 6 SEC.

The assessment of these conjugates in the HIV-Ag assay (FIGS. 7 A to F) showed that for E and D IgG(P6) ruthenium conjugates were superior to ruthenium conjugates from chemically polymerized antibody fragments (ori), IgG(P4) and IgG(P8) formats. S/N ratios from 6D7 and E IgG(P6)-Ru were 31% and 86% higher than the assay supplied with original reagent. Additionally an assay supplied with a bispecific multivalent IgG(P8) reagent with four binding antigen sites from clone D and four from E showed 18% better s/n than the original that contains oligomerized antibody fragments from D and E.

TABLE 10

| Calibration | Original | | | |
|---|---|---|---|---|
| for p24; | detection reagent | | | |
| Cal2/blank | (oligomers) | IgG(P4) | IgG(P6) | IgG(P8) |
| D epitope, | 100.00% | 67% | 131% | 72% |
| only | (D only) | | | |
| E epitope, | 100.00% | 105% | 186% | 121% |
| only | (E only) | | | |
| D and E | 100.00% | not tested | not tested | 118% |
| combined | (D and E | | | |
| | combined) | | | |

IgG(P4), IgG(P6) and IgG(P8) antibodies from HIV-ag Elecsys-Assay were labeled with Ruthenium at optimal label to protein ratio. Elecsys-HIV-Ag Assays were run with these IgG(P4), IgG(P6) and IgG(P8)-Ruthenium conjugates in parallel to the corresponding assay with current original conjugates (chemically crosslinked oligomers). HIV-Ag assay consists of two ruthenylated and polymerized antibodies (E and D). Each of these were tested and compared with new multivalent variants individually (A and B) or both were replaced by a E/D multivalent biclonal variant. Signal to noise (s/n) values were calculated and normalized.

1. A non-chimeric or chimeric multivalent recombinant antibody, wherein the antibody comprises p light chain polypeptides Fab_{L} and a dimer of two heavy chain polypeptides, wherein each heavy chain polypeptide has a structure of Formula I
_{N-terminus}[Fab_{H}-L-]ₙFab_{H}-L-dd(Fc_{H})[-L-Fab_{H}]_{m C-terminus} (Formula I)
wherein
(a) p is a value selected from the group consisting of 6, 8, and 10,
the value of p equals (2+2*(n+m)), and
each of m and n is selected independently from an integer of 1 to 3;
(b) “-” is a covalent bond within a polypeptide chain;
(c) each L is optional and, if present, is an independently selected variable linker amino acid sequence;
(d) each dd(Fc_{H}) is a heavy chain dimerization region of a heavy chain of a non-antigen binding immunoglobulin region;
(e) in the dimer the two dd(Fc_{H}) are aligned with each other in physical proximity;
(f) each Fab_{H} is independently selected from A_{H} and B_{H}, wherein A_{H} and B_{H} are different, and A_{H} and B_{H} are independently selected from the group consisting of
_{N-terminus}[VH-CH1]_{H C-terminus} (Formula II),
_{N-terminus}[VH-CL]_{H C-terminus} (Formula III),
_{N-terminus}[VL-CL]_{H C-terminus} (Formula IV), and
_{N-terminus}[VL-CH1]_{H C-terminus} (Formula V),
wherein
VH is a immunoglobulin heavy chain variable domain,
VL is a immunoglobulin light chain variable domain,
CH1 is a immunoglobulin heavy chain constant domain 1, and
CL is a immunoglobulin light chain constant domain;
(g) each Fab_{L} is independently selected from A_{L} and B_{L}, wherein A_{L} and B_{L} are different, and A_{L} and B_{L} are independently selected from the group consisting of
_{N-terminus}[VH-CH1]_{L C-terminus} (Formula VI),
_{N-terminus}[VH-CL]_{L C-terminus} (Formula VII),
_{N-terminus}[VL-CL]_{L C-terminus} (Formula VIII), and
_{N-terminus}[VL-CH1]_{L C-terminus} (Formula IX);
(h) each antigen binding site Fab_{H}:Fab_{L} of the antibody is an aligned pair, the alignment being signified by “:”, wherein each aligned pair is independently selected from the group consisting of A_{H}:A_{L} and B_{H}:B_{L}, wherein A_{H}:A_{L} and B_{H}:B_{L} are selected independently from the group consisting of
[VL-CH1]_{H}:[VH-CL]_{L},
[VL-CL]_{H}:[VH-CH1]_{L},
[VH-CH1]_{H}:[VL-CL]_{L}, and
[VH-CL]_{H}:[VL-CH1]_{L},
and wherein in each aligned pair the respective CL and CH1 are covalently linked via a disulfide bond.

2. The multivalent recombinant antibody according to claim 1, wherein the antibody is bispecific or monospecific.

3. The multivalent recombinant antibody according to claim 1, wherein the antigen binding site of A_{H}:A_{L} is capable of binding to a first epitope and the antigen binding site B_{H}:B_{L} is capable of binding to a second epitope, wherein the first and the second epitopes are different.

4. The multivalent recombinant antibody according to claim 3, wherein the antibody is bispecific and a first antigen binding site is capable of specifically binding to a first epitope, and a second antigen binding site is capable of specifically binding to a different second epitope, wherein the first epitope and the second epitope are comprised in a single molecule.

5. The multivalent recombinant antibody according to claim 3, wherein the antibody is bispecific and a first antigen binding site is capable of specifically binding to a first epitope, and a second antigen binding site is capable of specifically binding to a different second epitope, wherein the first epitope is comprised in a first molecule and the second epitope is comprised in a second molecule.

6. The multivalent recombinant antibody according to claim 5, wherein the first and the second molecules are identical or non-identical.

7. The multivalent recombinant antibody according to claim 5, wherein the two molecules are comprised in an aggregate or in a complex.

8. The multivalent recombinant antibody according to claim 1, wherein the antigen binding site of A_{H}:A_{L} is capable of binding to the same epitope as the antigen binding site B_{H}:B_{L}.

9. The multivalent recombinant antibody according to claim 8, wherein the antibody is monospecific and the antigen binding sites are identical or different.

10. The multivalent recombinant antibody according to claim 9, wherein the antigen binding sites are capable of specifically binding to an epitope comprised in a single molecule or in different molecules.

11. A kit of parts for the detection of an antigen, the kit comprising a multivalent recombinant antibody according to claim 1.

12. The kit according to claim 12, wherein the kit further comprises a detectable label.

13. A method for detecting an antigen, the method comprising the steps of contacting a multivalent recombinant antibody according to claim 1 with the antigen, thereby forming a complex of antigen and the multivalent recombinant antibody, followed by detecting formed complex, thereby detecting the antigen.

14. The method according to claim 13, the method comprising the steps of
(a) mixing a multivalent recombinant antibody according to claim 1 with a liquid sample suspected of containing the antigen,
(b) incubating the sample and the multivalent recombinant antibody of step (a), thereby forming a complex of antigen and the multivalent recombinant antibody if antigen is present and accessible for contact with the multivalent recombinant antibody during the incubation,
(c) detecting complex formed in step (b),
thereby detecting the antigen.

15. The method according to claim 13, the method comprising the steps of
(a) mixing a multivalent recombinant antibody according to claim 1 with a liquid sample suspected of containing the antigen,
(b) incubating the sample and the multivalent recombinant antibody of step (a), thereby forming a complex of antigen and the multivalent recombinant antibody if antigen is present and accessible for contact with the multivalent recombinant antibody during the incubation,
(c) immobilizing complex formed in step (b), and
(d) detecting immobilized complex,
thereby detecting the antigen.

16. A method according to claim 13, the method comprising the steps of
(a) mixing a labeled multivalent recombinant antibody according to claim 1 with a liquid sample suspected of containing the antigen,
(b) incubating the sample and the labeled multivalent recombinant antibody of step (a), thereby forming a complex of antigen and the labeled multivalent recombinant antibody if antigen is present and accessible for contact with the labeled multivalent recombinant antibody during the incubation,
(c) immobilizing complex formed in step (b), and
(d) detecting immobilized label,
thereby detecting the antigen.

17. A method according to claim 13, the method comprising the steps of
(a) adding a labeled multivalent recombinant antibody according to claim 1 to a solid phase suspected of containing the antigen on its surface,
(b) incubating the solid phase and the labeled multivalent recombinant antibody of step (a), thereby forming a complex of antigen and the labeled multivalent recombinant antibody if antigen is present and accessible for contact with the labeled multivalent recombinant antibody during the incubation, followed by
(c) washing the solid phase, thereby removing not complexed labeled multivalent recombinant antibody, followed by
(d) detecting label on the solid phase,
thereby detecting the antigen.

18. The method according to claim 17, wherein the solid phase is capable of capturing the antigen, and prior to step (a) a step of contacting the solid phase with a liquid sample suspected of containing the antigen is performed, wherein antigen is captured by the solid phase if antigen is present and accessible for capture by the solid phase.
